(19) United States
(12) Patent Application Publication
(10) Pub. No.: US 2020/0267945 A1
(43) Pub. Date: Aug. 27, 2020

(54) COMMUNICATION AND CONTROL SYSTEMS AND METHODS FOR MONITORING INFORMATION ABOUT A PLURALITY OF BEEHIVES

(71) Applicant: The Bee Corp., Bloomington, IN (US)

(72) Inventors: Ellie Symes, Bloomington, IN (US); Simon Kuntz, Bloomington, IN (US); Wyatt Wells, Bloomington, IN (US)

(21) Appl. No.: 16/499,662

(22) PCT Filed: Apr. 2, 2018
(86) PCT No.: PCT/US18/25715

§ 371 Date: Sep. 30, 2019

Related U.S. Application Data
(60) Provisional application No. 62/479,743, filed on Mar. 31, 2017.

(51) Int. Cl. A01K 47/06; G16H 40/67; G05B 15/02; G08B 25/00

(52) U.S. Cl. CPC A01K 47/06; G08B 25/00; G05B 15/02; G16H 40/67

(57) ABSTRACT

Technologies for monitoring beehives in an apiary includes one or more sensors communicatively coupled to a remote data analysis computing device, which is communicatively coupled to a client computing device. The sensors are configured to monitor beehives and/or the environment of the apiary and transmit the sensed data to the remote data analysis computing device. The remote data analysis computing device is configured to analyze the received sensor data, determine whether to perform an action as a function of a result of the analysis, and perform the function, as applicable. The client computing device is configured to interface with the remote data analysis computing device such that a user of the client computing device can view results of the data analysis and actions, as well as provide input and adjust settings administered by the remote data analysis computing device. Other embodiments are described herein.

### BACKGROUND OF THE DISCLOSED EMBODIMENTS

Honeybee populations have been in decline since the 1980s, and the term “Colony Collapse Disorder,” was coined in 2007 to describe the troubling state of the bees. Beekeepers in the past could rely on 10-15% hive losses (i.e., bee mortality rates) each year, which was perceived as a sustainable loss percentage. Today, beekeepers are experiencing an average hive loss rate of 30% each year, with hive loss rates reaching nearly 50% in some regions, which has resulted in a fairly significant decrease in honey production over the last couple of decades. Diverse beekeeping beliefs (i.e., decision-making regarding hive management) have contributed to the hive loss rates, typically with little to no actual data being used to support hive management practices.

Additionally, beekeepers generally do not keep an accurate accounting of the information associated with their apiaries (e.g., the condition of the hives, bee counts, environment conditions, losses, etc.). Further, current research on honeybees relies on self-selection citizen science, and is limited to the qualitative information supplied by beekeepers, which, as noted, can be largely inaccurate. In this field, there is a general lack of quantitative data on the ecology and production efficiency of apiaries and the bees/hives contained therein. Therefore, there is a need for improved technologies for monitoring and analyzing data of beehives in an apiary.

### SUMMARY OF THE DISCLOSED EMBODIMENTS

In one embodiment, a communications and control system and method for monitoring information about a plurality of beehives is provided. In one aspect of an embodiment of such a system, information or data is measured or otherwise observed, such as using suitable sensors, regarding the bees, and/or the beehives, and/or the environment around the beehives, or other relevant data or information. Such information measured may include one or more of temperature(s) at different points on or in the hive, gas (such as oxygen, carbon monoxide, and/or carbon dioxide gases) or other material or toxin levels, infrared or other wavelength information, weight and/or mass, activity level, decibel or other noise level measures, humidity levels, pressure, activity such as may be measure by movement or another visual measure, GPS or similar data regarding physical location, altitude, orientation or relative orientation, and other information or data as would occur to one of ordinary skill in the art regarding the beehive, bees, their activity, health, stress or other beehive effects.

In one embodiment, the sensors sense and transmit or store the data or information. Information may be stored at the beehive for later use, processing, or collection. The data or information may be collected through a variety of methods using a variety of systems and components well known in the art, such as automated or manual collection, pushing the information using a transmitter located at the beehive. Such data or information may be processed at the beehive or may be processed using devices or systems as disclosed in this disclosure or as would be known to one of ordinary skill in the art. The information may be processed at one or more of at the beehive, at another beehive that is in the same apiary or is separate from the beehive, and using other devices or systems remote from the beehives.

Other information and data can also be manually gathered or input, accessed, observed or collected relating to environmental conditions at or near each beehive and/or group of beehives. Such information may include the information described above, and/or other information such as wind speed and/or direction, precipitation type and amount, crop information (such as crop type and related data, plant location, plant density, crop yield information whether aggregate or by location in an orchard, field or plot), pesticide, fertilizer, and/or irrigation type, quantity and rate information, planting and harvest information, and other agricultural information. Such information and other data may be collected or accessed through other systems (such as farm implements and separate agricultural monitoring systems on the farm or orchard, or from other available databases or measurement/monitoring systems).

In one embodiment, the system and method may be configured to perform calculations and analyses. Calculations and analyses may be manual, automated, and may be performed using manually developed functions, predetermined or established functions, or machine learning. Such calculations and analyses may include for example recommended density or relative location of beehives placed for pollination in an orchard or in a field or plot, effectiveness or net yield change in a given crop, pesticide and/or fertilizer usage as a function of hive health, activity or other beehive information,

In one embodiment, the system and method may be configured to generate and/or communicate reports. Such reports may include graphs, charts, lists, and other displays known to those of ordinary skill in the art. Such reports may include data, analyzed data, results of calculations and other derived information from the data and information gathered or accessed as described herein.

In one embodiment, the system and method may be configured to generate and communicate suggestions, recommendations, or instructions such as physical movement of one or more beehives to a new and/or particular location in an apiary or group of beehives placed for pollination or relative to a portion of crops, replacement of a beehive, collection of honey in a beehive, replacement of a queen, inspection, repair, or maintenance of hive structural or sensor or other components as disclosed herein, and other actions to be taken whether automatically or manually by a famer, worker or beekeeper, and whether directly or remotely. These suggestions may be based on observed data and information gathered or may be based on analyses or calculations of such data.

In one embodiment, the system and method may be configured to generate and communicate actions. Such actions can be communicated by or within parts of the system, including those disclosed herein or as would be understood by one of ordinary skill in the art, and may illustratively include such actions as changing beehive orientation, activating or positioning windscreens, activating or positioning sunscreens, opening ventilation equipment or doors, activating smoke, water, food, nutrients, medication, or other devices for providing a supply to the bees in or around the beehive.

In one aspect, a method for monitoring and analyzing data of beehives in an apiary includes receiving, by a remote data analysis computing device, sensor data from one of a plurality of sensors associated with the apiary; analyzing, by the remote data analysis computing device, the received data; determining, by the remote data analysis computing device, whether to perform an action as a function of a result of the analysis; and performing, by the remote data analysis computing device and in response to a determination the action is to be performed, the action.

In some embodiments, analyzing the sensor data comprises to analyze the data using one or more machine learning algorithms. In other embodiments, analyzing the sensor data comprises to analyze the data using one or more regression analysis methods. In still other embodiments, the method further comprising aggregating, by the remote data analysis computing device, the received data with previously received sensor data from at least a portion of the plurality of sensors, wherein analyzing the received data includes analyzing the aggregated data.

In some embodiments, the method further comprising comparing, by the remote data analysis computing device, the result of the analysis to one or more threshold values, wherein determining whether to perform the action as a function of the result of the analysis comprises determining whether to perform the action as a function of the comparison. In some embodiments, performing the action in response to a determination the action is to be performed comprises performing the action in response to a determination the result of the analysis meets or violates one or more threshold values. In other embodiments, performing the action comprises transmitting a message to a corresponding data monitoring application, wherein the message includes information related to the action that is usable to display to a user of the corresponding data monitoring application. In still other embodiments, performing the action comprises transmitting a command to an actuator of the apiary, wherein the command is usable by the actuator to adjust a position of a mechanism attached to the actuator. In yet other embodiments, performing the action comprises transmitting an email to an email address, wherein the email includes information related to the action that is usable to indicate to notify a user of the action. In still yet other embodiments, performing the action comprises transmitting a text message to a phone number, wherein the text message includes information related to the action that is usable to notify a user of the action.

### BRIEF DESCRIPTION OF THE DRAWINGS

The embodiments and other features, advantages and disclosures contained herein, and the manner of attaining them, will become apparent and the present disclosure will be better understood by reference to the following description of various exemplary embodiments of the present disclosure taken in conjunction with the accompanying drawings, wherein:

FIG. 1 is a schematic block diagram of a system for using sensor data to monitor beehives in an apiary that includes multiple sensors located within the apiary which are communicatively coupled to a remote data analysis computing device that is communicatively accessible by a client computing device;

FIG. 2 is a block diagram of an illustrative embodiment of the computing devices of the system of FIG. 1;

FIG. 3 is a block diagram of an illustrative embodiment of an environment of the client computing device of the system of FIG. 1;

FIG. 4 is a block diagram of an illustrative embodiment of an environment of the remote data analysis computing device of the system of FIG. 1; and

FIG. 5 is a schematic flow diagram of a method for monitoring and analyzing data of beehives in the apiary of the system of FIG. 1.

### DETAILED DESCRIPTION OF THE DISCLOSED EMBODIMENTS

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

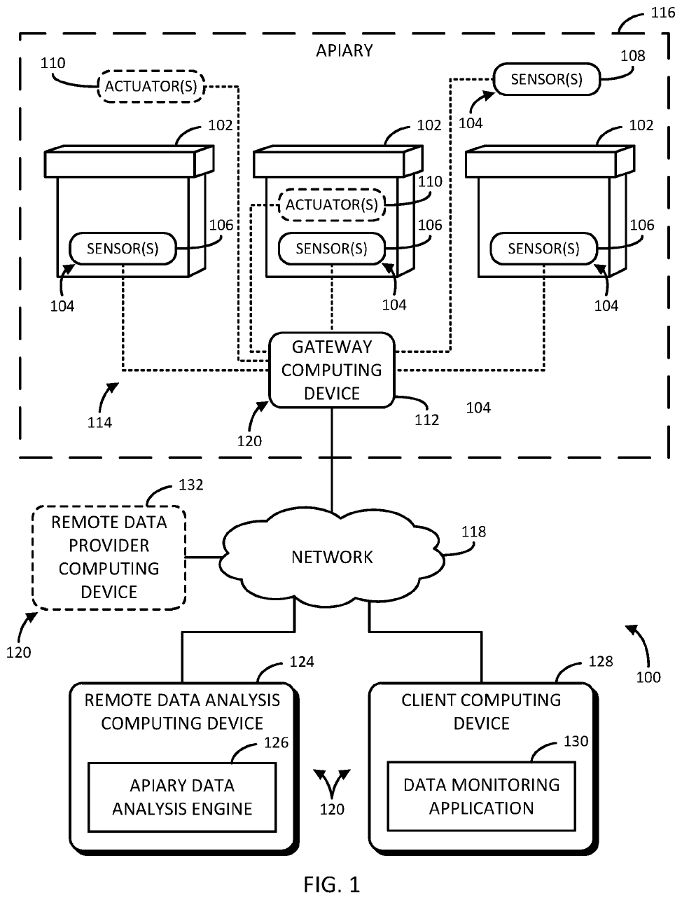

FIG. 1 illustrates a system 100 for monitoring an apiary 116, or “bee yard,” that includes a number of beehives 102 using multiple sensors 104 located within the apiary 116. Although the term bee yard or apiary is used herein, it is understood that the beehives in this disclosure do not have to be of a single apiary or in any close proximity to one or more other beehives of the system or method, and that the term apiary is intended to cover any number of one or more beehives that are to be monitored and part of the system and method described herein.

As illustratively shown, each of the sensors 104 are communicatively coupled to one or more computing devices 120 either directly or over a network 118. The illustrative computing devices 120 include a gateway computing device 112, a remote data analysis computing device 124, a client computing device 128, and a remote data provider computing device 132. It should be appreciated that additional and/or alternative computing devices 120 may be included in other embodiments.

The sensors 104 are configured to collect data of the environment in which they are deployed (e.g., internal or external to the beehives 102) and transmit (e.g., via messages, network packets, etc.) the collected data to the remote data analysis computing device 124. To do so, the sensors 104 are configured to transmit the collected data to the gateway computing device 112 over a wireless, cell, satellite, or other suitable network 114. In turn, and the gateway computing device 112 is configured to forward the collected data received from the sensors 102 to the remote data analysis computing device 124 via the network 118.

As will be described in further detail below, the remote data analysis computing device 124 is configured to monitor the data collected by and received from the sensors 104 to determine whether a condition exists such that an action should be taken (e.g., harvesting the produced honey from one or more of the beehives 102, attaching one or more supers to one or more of the beehives 102, moving/placing/repairing/replacing one or more of the beehives 102, adjusting a feeding schedule, etc.). In an illustrative example, the remote data analysis computing device 124 may be configured to determine locational placement of one or more of the beehives 102 within the apiary 116 in an effort to maximize honey production efficiency and/or reduce bee mortality rates.

The beehives 102 may be embodied as any type of enclosed structure in which a colony of honey bees is intended to live within. The beehives 102 may be configured as any type of artificial beehive 102, typically based on regional locale, such as, but not limited to, a top-bar hive, Warre (European style) hive, Langstroth (stacked) hive, etc. It should be appreciated that the beehives 102 as described herein are intended to be used such that honey produced from the bees living therein may be extracted therefrom. It should be further appreciated that, in some embodiments, the apiary 116, and more particularly the beehives 102 contained therein, may be intended to serve one or more additional and/or alternative purposes, such as the pollination of nearby crops, to provide safe-housing to mitigate the effects of colony collapse disorder, etc.

Each of the sensors 104 may be embodied as any combination of firmware, software, hardware, and/or circuitry capable of performing the functions described herein, including detecting a present condition and/or events/changes in the environment in which the sensors 104 are deployed. The illustrative sensors 104 include beehive sensors 106 and environment sensors 108. It should be appreciated that for the purposes of the description provided herein, the beehive sensors 106 include any type of sensor 104 usable to detect a state or condition of the beehive 102. In other words, beehive sensors 106 include any types of sensors 104 capable of measuring data (i.e., beehive data) related to a respective beehive 102 to which it is attached or in proximate location thereof. Examples of such beehive data may include a weight of the beehive 102, a temperature inside the beehive 102, an activity level inside the beehive 102, etc.

It should be further appreciated that for the purposes of the description provided herein, the environment sensors 108 include any type of sensor 104 usable to detect a state or condition of an environment condition the apiary 116. In other words, the environment sensors 108 include any types of sensors 104 usable for measuring data (i.e., environment data) of the apiary 116 in which the beehives 102 reside, or the immediately surrounding area of the apiary 116. Examples of such environment data may include weather conditions (e.g., temperature, wind, humidity, sunlight, shade, UV levels, etc.) of the apiary at a given point in time and/or over a particular window of time, a time of day (e.g., dawn, dusk, etc.), detected motion/activity (e.g., of beekeepers, bees, animals, etc.), etc.

In some embodiments, the system 100 may additionally include one or more actuators 110. The actuators 110 may be embodied as any combination of firmware, software, hardware, and/or circuitry capable of performing the functions described herein, such as moving or controlling a mechanism or component. As illustratively shown, one or more of the actuators 110 may be located proximate to one of the beehives 102. In other words, in some embodiments, one or more of the actuators 110 may be affixed to a mechanism or component internal to or otherwise attached to one of the beehives 102. Additionally or alternatively, as also illustratively shown, one or more of the actuators 110 may be affixed to a mechanism or component located external to the beehives 102, in some embodiments. In an illustrative example, one of the actuators 110 may be configured to open a valve to release a variable amount of smoke from a smoker to which the respective actuator 110 has been affixed.

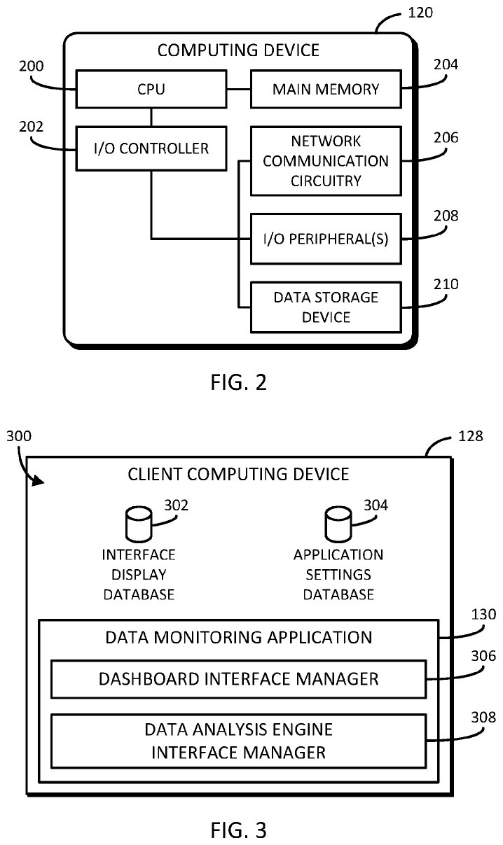

The computing devices 120 may be embodied as any combination of firmware, software, hardware, and/or circuitry capable of performing the functions described herein. Referring now to FIG. 2, an embodiment of an illustrative computing device 120 is shown. The illustrative computing device 120 includes a CPU 200, an input/output (I/O) controller 202, a memory 204, a network communication circuitry 206, one or more I/O peripherals 208, and a data storage device 210. In some embodiments, one or more of the illustrative components may be combined on a single system-on-a-chip (SoC) on a single integrated circuit (IC). It should be appreciated that alternative embodiments may include additional, fewer, and/or alternative components to those of the illustrative computing device 120, such as may be found in a typical computing device (e.g., a graphics processing unit (GPU), a power supply, etc.). It should be further appreciated that the components and capabilities of each of the computing devices 120 may be based on the functionality of the respective computing devices as described herein.

The CPU 200 may be embodied as any type of hardware or combination of circuitry capable of processing data. Accordingly, the CPU 200 may include a single processing core (e.g., in a single-core processor architecture) or more than one processing core (e.g., in a multi-core processor architecture). Irrespective of the number of processing cores, the CPU 200 is capable of reading and executing program instructions. In some embodiments, the CPU 200 may include a cache memory (not shown). In such embodiments, the cache memory may be integrated directly with the CPU 200 or placed on a separate chip with a separate interconnect to the CPU 200. It should be appreciated that, in some embodiments, pipeline logic may be used to perform software and/or hardware operations (e.g., network traffic processing operations), rather than commands issued to/from the CPU 200.

The I/O controller 202, or I/O interface, may be embodied as any type of computer hardware or combination of circuitry capable of interfacing between input/output devices and the computing device 120. Illustratively, the I/O controller 202 is configured to receive input/output requests from the CPU 200, and send control signals to the respective input/output devices, thereby managing the data flow to/from the computing device 120.

The memory 204 may be embodied as any type of computer hardware or combination of circuitry capable of holding data and instructions for processing. Such memory 204 may be referred to as main or primary memory. It should be appreciated that, in some embodiments, one or more components may have direct access to memory, such that certain data may be stored via direct memory access (DMA) independently of the CPU 200.

The network communication circuitry 206 may be embodied as any type of computer hardware or combination of circuitry capable of managing network interfacing communications (e.g., messages, datagrams, packets, etc.) via wireless and/or wired communication modes. Accordingly, in some embodiments, the network communication circuitry 206 may include a network interface controller (NIC) capable of being configured to connect the computing device 120 to a computer network (e.g., the network 106).

The one or more I/O peripherals 208 may be embodied as any auxiliary device configured to connect to and communicate with the computing device 120. For example, the I/O peripherals 208 may include, but are not limited to, a touchpad, a keypad, a keyboard, a microphone, one or more buttons, a display (e.g., a liquid crystal diode (LED) display), a touchscreen, a speaker, one or more illuminating components (e.g., light emitting diode(s) (LEDs)), etc. Accordingly, it should be appreciated that some I/O peripherals 208 are capable of facilitating only input or output functions, while others are capable of performing both functions (i.e., input and output).

The data storage device 210 may be embodied as any type of computer hardware capable of the non-volatile storage of data (e.g., semiconductor storage media, magnetic storage media, optical storage media, etc.). Such data storage devices 210 are commonly referred to as auxiliary or secondary storage, and are typically used to store a large amount of data relative to the memory 204 described above.

Referring back to FIG. 1, the gateway computing device 112 may be embodied as any type of network gateway device capable of forwarding network traffic between networks (e.g., the wireless network 114 and the network 118). It should be appreciated that the type and functional capacity of the gateway computing device 112 may be dependent on the type of the wireless network 114 in which the gateway computing device 112 is used. The wireless network 114 may be embodied as any type of network architecture/topology capable of performing the functions described herein. For example, in some embodiments, the wireless network 114 may be configured as a star network, in which the gateway computing device 112 serves as a central hub (see, e.g., the illustrative wireless network 114 of FIG. 1). In other words, in the star network configuration, each of the sensors 104 are linked to the gateway computing device 112 only, not to one or more of the other sensors 104.

In an alternative embodiment, the wireless network 114 may be configured as a mesh network, such as a wireless sensor network, in which each of the sensors 104 include repeater/routing capabilities. Accordingly, in the mesh network configuration, unlike the star network configuration, each of the sensors 104 are connected to one or more of the other sensors 104 of the wireless network 114. As such, information from one of the sensors 104 can be relayed onto another of the one or more sensors 104 in an effort to transmit collected data to the gateway computing device 112.

The network 118 may be implemented as any type of wired and/or wireless network, such as a local area network (LAN), a wide area network (WAN), a global network (e.g., the Internet), etc. Accordingly, one or more network communication technologies (e.g., Ethernet, cellular, satellite, etc.) and associated protocols may be used. The network 118 may include one or more communicatively coupled network computing devices (not shown) for facilitating the flow and processing of network communication traffic via a series of interconnects. Such network computing devices may include, but are not limited to, one or more virtual and/or physical access points, routers, switches, servers, network hubs, compute devices, storage devices, etc.

It should be appreciated that one or more of such network computing devices may be configured to couple one or more of the computing devices 120 (e.g., the gateway computing device 112, the remote data analysis computing device 124, the client computing device 128, and the remote data provider computing device 132 of the system 100 of FIG. 1) to the network 118 as needed to facilitate network-based communications therebetween. To do so, the computing devices 120 may connect to the network 118 (e.g., via the network communication circuitry 206 of FIG. 2) using wired (e.g., Ethernet, token ring, etc.) and/or wireless (e.g., Bluetooth®, Wi-Fi®, wireless broadband, ZigBee®, etc.) communication technologies and associated protocols.

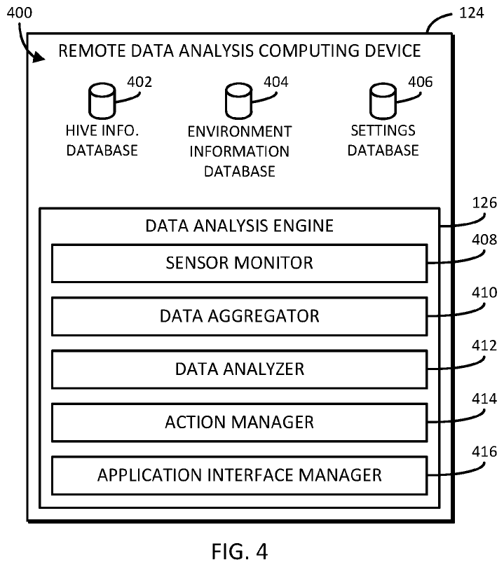

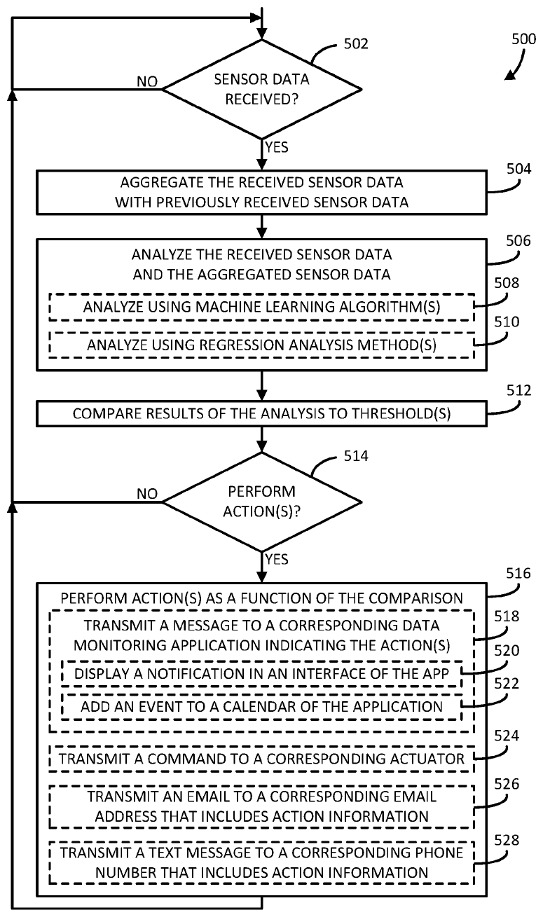

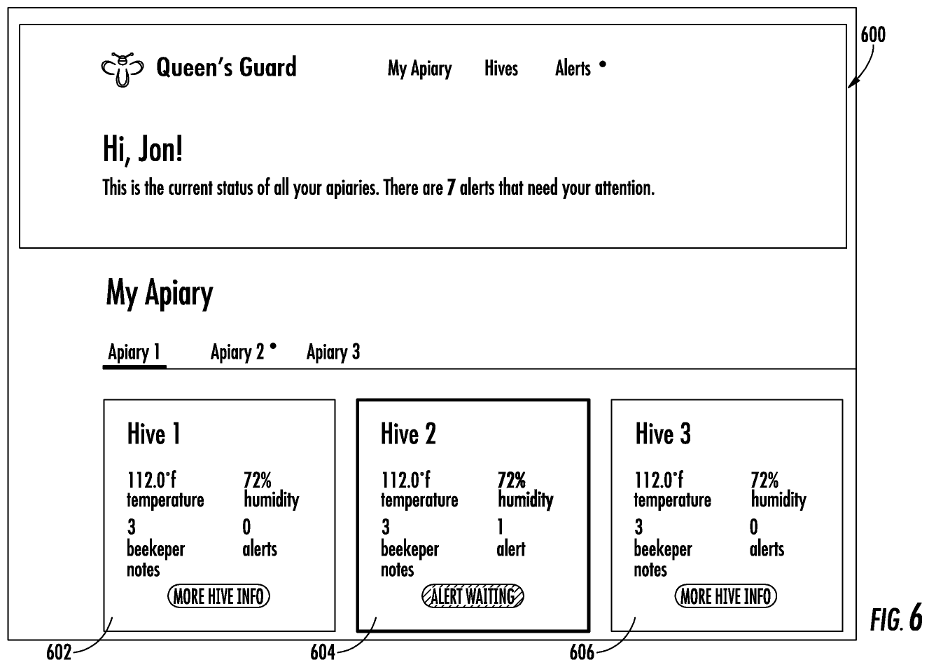

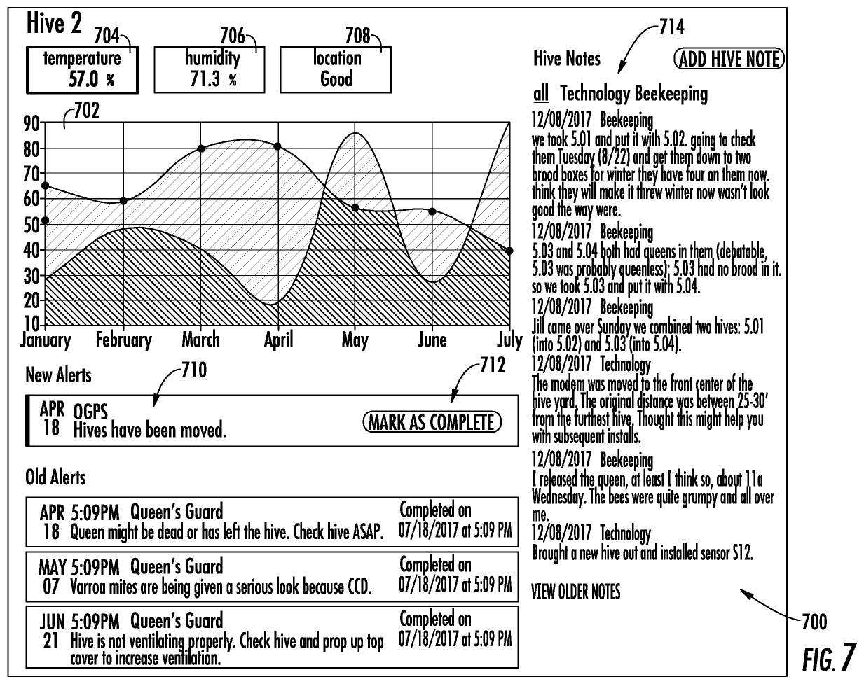

The remote data analysis device 124 may be embodied as any type of computing device 120 capable of monitoring, aggregating, and storing received data (e.g., data collected and transmitted by the sensors 104 of the apiary 116), as well as analyzing the data to determine whether an action should be taken as a result of the analysis. To do so, the illustrative remote data analysis device 124 includes or is otherwise communicatively coupled to an apiary data analysis engine 126 (see, e.g., the illustrative apiary data analysis engine 126 of FIG. 4). The apiary data analysis engine 126, which is described in further detail below, may be embodied as a web server program (e.g., in a client-server architecture) running on the remote data analysis device 124 (e.g., as a cloud-based service). The apiary data analysis engine 126 is additionally configured to communicate with the data monitoring application 130 of the client computing device 128 (e.g., in a client-server relationship via the network 118).

The remote data analysis device 124 may be embodied as any type of computing device with sufficient storage and compute capabilities to perform the functions described herein. It should be appreciated that while the remote data analysis device 124 is illustratively shown as a single computing device 120, the remote data analysis device 124 may include more than one computing device 120, in some embodiments, such as in distributed or parallel computing embodiments. For example, in some embodiments, the remote data analysis device 124 may be embodied as one or more servers (e.g., stand-alone, rack-mounted, etc.) and/or combination of compute blades and data storage devices (e.g., of a storage area network (SAN)).

It should be appreciated that the remote data analysis device 124 may be communicatively coupled to more than one apiary (e.g., via a respective gateway computing device 112 of each apiary to which the remote data analysis device 124 is communicatively coupled) at any given point in time. In other words, the remote data analysis device 124 may be configured to monitor, aggregate, store, and analyze the data corresponding to more than one apiary 116 at a time. Accordingly, in such embodiments, the remote data analysis device 124 may be configured to leverage data from one or more apiaries 116 to analyze another apiary 116.

The client computing device 128 may be embodied as any type of computing device 120 capable of displaying data received from the remote data analysis device 124, receiving input data from a user of the client computing device 128, and transmitting the received input data to the remote data analysis device 124. To do so, the illustrative client computing device 128 includes a data monitoring application 130 (see, e.g., the illustrative data monitoring application 130 of FIG. 3). The data monitoring application 130, which is described in further detail below, may be embodied as a user interfacing thin-client application, such as, but not limited to, a web browser, a web application, a mobile application, an application programming interface (API) infrastructure, or other Internet connected software application. It should be appreciated that the data monitoring application 130 is configured to run, or otherwise being executed on, the client computing device 128 and is additionally configured to communicate with the apiary data analysis engine 126 of the remote data analysis device 124 (e.g., in a client-server relationship via the network 118).

In some embodiments, the client computing device 128 may be embodied as a mobile computing device that uses mobile-specific hardware and software (e.g., an operating system) for operating, executing, and providing services and applications to support a mobile architecture and portability. Illustrative examples of such mobile computing devices may include, but are not limited to, smartphones, wearables (e.g., smartwatches, smart glasses, etc.), tablets, laptops, notebooks, etc. Alternatively, in other embodiments, the client computing device 128 may be a stationary computing device, such as a desktop computer, an all-in-one computer, etc. It should be appreciated that, in some embodiments, the system 100 may include more than one client computing device 128. In other words, more than one client computing device 128 may be communicatively coupled to the remote data analysis device 124 at a given point in time.

The remote data provider computing device 132 may be embodied as any type of computing device capable of performing the functions described herein, including, but not limited to, aggregating data and transmitting the aggregated data, or a result of an analysis performed thereon, to the remote data analysis device 124. For example, the remote data provider computing device 132 may be configured to collect and/or aggregate weather related data and transmit present and projected future weather conditions. Accordingly, the remote data provider computing device 132 may be embodied as a server, a compute device, a storage device, or a combination thereof. It should be appreciated that, similar to the remote data analysis device 124, the remote data provider computing device 132 may include more than one computing device 120, in some embodiments, such as in distributed or parallel computing embodiments.

Referring now to FIG. 3, in an illustrative embodiment, the client computing device 128 establishes an environment 300 during operation. The illustrative environment 300 includes an interface display database 302 and an application settings database 304, each of which may be stored in a memory and/or data storage device of the client computing device 128. It should be appreciated that, in some embodiments, the data stored in, or otherwise represented by, each of the interface display database 302 and the application settings database 304 may not be mutually exclusive relative to each other.

The interface display database 302 is configured to store interface display information received from the remote data analysis computing device 124. Such interface display information may be usable to display or render/display a user interface of the data monitoring application to a display of the client computing device 128. The application settings database 304 is configured to store settings information of the data monitoring application 130. The settings information may include any settings usable by the data monitoring application 130 to determine which data types are being monitored, define one or more thresholds, set a weight and/or priority level to certain types of data, and/or set one or more actions associated with the collected data and corresponding thresholds. For example, the settings may include user-definable thresholds and/or parameters that may be usable to dynamically adjust one or more thresholds.

The illustrative environment 300 additionally includes an illustrative embodiment of the data monitoring application 130. The illustrative data monitoring application 130 includes a dashboard interface manager 306 and a data analysis engine interface manager 308. It should be appreciated that the interfaces described herein for managing the user interactions (e.g., displaying the GUI and receiving user inputs) are graphical user interfaces (GUIs) which allow the user to interact with the application referred to herein through GUI elements, graphical images, and visual indicators. Accordingly, the dashboard interface manager 306 is configured to render/display a GUI of the data monitoring application 130 to a display of the client computing device 128. In some embodiments, at least a portion of the user interface of the data monitoring application 130 may be viewed and/or accessed as a function of a level of permission(s) and/or needs of the user. For example, the dashboard interface manager 306 may be configured to display certain information (e.g., via dedicated interface modules) based on whether the user is a beekeeper, a farmer, a researcher, etc., such that the respective user type can access the information and adjust settings relevant to that particular user type.

In some embodiments, the dashboard interface manager 306 may be additionally configured to generate audible tones (i.e., initiate playback of a sound file), such as may be associated with a notification message, or some other indication of an event associated with the data monitoring application 130. The dashboard interface manager 306 is further configured to receive inputs from a user of the client computing device 128, such as may be received via a touchscreen display of the client computing device 128, or other peripheral device (e.g., stylus, mouse, keyboard, keypad, microphone, etc.) connected to the client computing device 128.

The data analysis engine interface manager 308 is configured to interface with the remote data analysis computing device 124. To do so, the data analysis engine interface manager 308 is configured to establish a communication channel with the remote data analysis computing device 124, which is usable to transmit and receive messages (e.g., network traffic, packets, messages, etc.) therebetween.

Accordingly, the data analysis engine interface manager 308 is configured to generate messages for transmission to the remote data analysis computing device 124, such as may be generated based on inputs received from a user by the dashboard interface manager 306. As such, the data analysis engine interface manager 308 can provide information (e.g., changed settings, dashboard display requests, initiated actions, etc.) that is usable by the remote data analysis computing device 124, and more particularly by the apiary data analysis engine 126, to update a setting associated with the data being monitored, adjust the analysis being performed, etc. Additionally, in some embodiments, the information may be usable by the remote data analysis computing device 124 to initiate an operation to be performed, or otherwise set a time/schedule for an operation to be performed, such as may be performed via one of the actuators 100 of the apiary 116. Additionally, in some embodiments, the occurrence of a condition (such as the actual or forecasted temperature drops below a threshold) may be usable by the remote data analysis computing device 124 to initiate an operation to be performed, or otherwise set a time/schedule for an operation to be performed, such as may be performed via one of the actuators 100 of the apiary 116. The data analysis engine interface manager 308 is additionally configured to process messages received from the remote data analysis computing device 124. For example, the data analysis engine interface manager 308 is configured interpret a message received from the remote data analysis computing device 124 to determine what information is to be displayed by the data monitoring application 130 to a display of the client computing device 128 (e.g., in a GUI dashboard of the data monitoring application 130) and/or other output device coupled to the client computing device 128. It should be appreciated that the received information may include data (e.g., in a payload of a network packet) that includes information related to an object that is usable to download and/or render one or more GUI elements, icons, graphics, text, etc., for output by the client computing device 128.

Referring now to FIG. 4, in an illustrative embodiment, the remote data analysis computing device 124 establishes an environment 400 during operation. The illustrative environment 400 includes a hive information database 402, an environment information database 404, and a settings database 406, each of which may be stored in a memory and/or data storage device of the remote data analysis computing device 124. The hive information database 402 may be configured to store beehive 102 related data (i.e., beehive data), such as may be captured by and received from the beehive sensors 106 of FIG. 1; whereas the environment information database 404 may be configured to store data related to the environment (i.e., environment data) of the apiary 116 in which the beehives 102 reside, such as may be captured by and received from the environment sensors 108 of FIG. 1.

It should be appreciated that, in some embodiments, the data stored in, or otherwise represented by, each of the hive information database 402, the environment information database 404, and the settings database 406 may not be mutually exclusive relative to each other. In some embodiments, one or more of the hive information database 402, the environment information database 404, and the settings database 406 may include data from beehives 102 of multiple apiaries 116. In such embodiments, the data from one apiary 116 may be siloed from and/or combined with the data from other apiaries 116.

The illustrative environment 400 additionally includes an illustrative embodiment of the apiary data analysis engine 126. The illustrative data analysis engine 126 includes a sensor monitor 408, a data aggregator 410, a data analyzer 412, an action manager 414, and an application interface manager 416. It should be appreciated that, in some embodiments, one or more of the components of the illustrative data analysis engine 126 may be combined to result in fewer components (e.g., the data analyzer 412 may perform the function of the sensor monitor 408 and/or the data aggregator 410). Additionally, it should be further appreciated that, in some embodiments, one or more of the components of the illustrative data analysis engine 126 may be divided into a subset of components.

The sensor monitor 408, which may be embodied as any type of firmware, hardware, software, circuitry, or combination thereof, is configured to receive the data collected by the sensors 104 and store the received sensor data in the appropriate fields of the respective database (e.g., the hive information database 402 and/or the environment information database 404). It should be appreciated that the types of beehive data collected from the beehive sensors 106 may be predicated on the types of beehive sensors 106 in use in a particular beehive 102. Similarly, it should be appreciated that the types of environment data collected from the environment sensors 108 may be predicated on the types of environment sensors 108 in use in a particular apiary 116.

As described previously, the beehive data may include any data related to at least a portion of a beehive 102, such as a weight of the beehive 102, a temperature inside the beehive 102, an activity level inside the beehive 102, pheromone levels, etc. As also described previously, the environment data of the apiary 116 may include weather conditions (e.g., temperature, wind, humidity, sunlight, shade, UV levels, etc.), a time of day (e.g., dawn, dusk, etc.), detected motion/activity, etc. Such data can be used to make decisions regarding and issue action instructions such as to close doors, change louvre positions, rotate the beehive, and the like.

In an illustrative example, one or more of the bees may be individually monitored, or tracked, using a micro-sized transponders/transceivers (e.g., a micro NFC/RFID transponder, a Bluetooth® Low Energy (BLE) micro module, or some other type of low power wireless communication technology module) that may be used for wireless tracking and/or identification purposes. In such embodiments, the micro-sized transponder may be configured to interface with one or more sensors 104 and/or readers associated with the micro-sized transponder (e.g., an RFID reader) that may be placed within the apiary 116, or more particularly within or otherwise associated with a particular beehive 102. For example, in such embodiments, ingress/egress pattern data may be captured for analysis.

Additionally or alternatively, in some embodiments, one or more GPS tracking devices may be employed. For example, in such embodiments, the queen bee of a particular beehive 102 may have a GPS tracking device affixed thereto such that movement, flight speed, flight pattern, and other properties can be tracked and monitored. Accordingly, in such embodiments, the sensor monitor 408 may be configured to interface with or otherwise be configured to interpret the tracking information received from such tracking technologies for analysis.

The data aggregator 410, which may be embodied as any type of firmware, hardware, software, circuitry, or combination thereof, is configured to aggregate, refine, and enhance the received data. To do so, the data aggregator 410 may be configured to gather data from a number of data sources other than the sensors 104. In other words, while the majority of the data described as being collected herein has been described in relation to the data received from the sensors 104, it should be appreciated that additional and/or alternative data elements may be received from a source other than the sensors 104, such that the externally received data may be usable to refine and/or enhance the sensor 104 received data.

For example, in some embodiments, the data aggregator 410 may be further configured to aggregate data received from a user (i.e., by way of the data monitoring application 130), such as may be received via the application interface manager 416, as well as any externally received and/or user input data. Accordingly, it should be appreciated that the hive information database 402 and/or environment information database 404 may include additional information that was not collected by the sensors 104, such as may have been acquired, gathered, or input by another source (e.g., a user of the data monitoring application 130 of the client computing device 128) or received from an external source (e.g., via an API of a data aggregator).

In an illustrative example, a user (e.g., a beekeeper, an apiary owner, an administrator, etc.) may input information (e.g., via the data monitoring application 130) usable by the data analysis engine 126 to refine and/or enhance the data of the hive information database 402 and/or environment information database 404 (i.e., the data that has been received from the beehive sensors 106 and/or the environment sensors 108). The user input data may include data related the bees in the apiary 116 or a particular one or more of the beehives 102.

For example, the user input data may include genetic information, breeding information, traits, wing span and thickness, coloration, etc. In another example, the user input data may be related to a particular hive, such as egg-laying levels, propolis levels, weight/size of individual bees in population (e.g., population size, number of drones, proportion of new/winter bees, proportion of workers to drones, etc.), honey production levels, parasite/pest types/levels (e.g., Varroa mites, small hive beetles, wax moths, etc.), travel routes/speed of the queen and/or other bee types, etc. In still another example, the user input data may be related to the feeding of the bees in the apiary 116 or a particular one or more of the beehives 102, such as a time of day of the feeding, a type/amount of food distributed, a type/amount of vitamin supplements added to the food, etc.

The user input data may additionally include data related a beekeeper prior to, during, or subsequent to interaction(s) with one or more beehives 102 of the apiary 116. Accordingly, it should be appreciated that such information may be input prior to, during, or subsequent to interaction(s), depending on the type of input. For example, the interaction data may include dietary information of the beekeeper, a mood of the beekeeper, a present health condition of the beekeeper, etc., prior to an interaction. Additionally and/or alternatively, the interaction data may include a duration of the interaction (e.g., with a particular beehive 102, in the apiary 116, etc.), a description of beekeeping attire worn (e.g., veils/masks, clothing, etc.), type of equipment used (e.g., smoker brand, size, fuel type, etc.) a mood of the beekeeper, noise(s) made, whether the beekeeper was stung (e.g., at a particular beehive 102, in the apiary 116, etc.), etc., during an interaction.

In another illustrative example, the data aggregator 410 may be configured to interface with an externally managed data source (e.g., an external data aggregator) to refine and/or enhance the data of the hive information database 402 and/or environment information database 404. Such externally acquired data may include weather prediction data (e.g., from a weather data aggregator), animal/insect migration patterns, air quality levels, etc. Additionally, the data aggregator 410 may be configured to collect external environmental data of one or more areas neighboring the apiary 116. Such neighboring environmental data may include, but is not limited to, a number and/or types of plants (e.g., food sources, trees, medicinal plants, etc.), other pollinators/apiaries, a number and/or types of animals/insects/humans, land type/usage, pesticide usage, water pollution levels, etc. It should be further appreciated that some input data may be discernable from other input data via an externally managed data source. For example, an address of the apiary 116 may be usable to determine an elevation, latitude and longitude, etc., of the apiary 116. GPS or other geo-location information and data, altitude, and orientation and other positioning information such as measured through accelerometers and other devices known to those of ordinary skill in the art.

While the data described herein is described as being collected from a particular source (e.g., beehive sensors 106, environment sensors 108, user input, 3^{rd} party data aggregators, etc.), it should be appreciated that the data may be collected from an alternative data source, which may or may not be described herein. For example, in some embodiments, at least a portion of the data described herein as being collected via user input may be collected by one or more sensors 104 in other embodiments.

The data analyzer 412, which may be embodied as any type of firmware, hardware, software, circuitry, or combination thereof, is configured to analyze the received data, as well as the aggregated data, such that correlations may be determined therefrom which are usable to identify one or more actions that may be taken as a function of the correlations and present condition information. To do so, the data analyzer 412 may be configured to use one or more machine learning algorithms, regression analysis methods, and/or the like to perform the analysis. The data analyzer 412 may be further configured to transmit the results of the analysis to the action manager 414 and/or the application interface manager 416, each of which are described in further detail below.

For example, the data analyzer 412 may be configured to compare at least a portion of a result of the analysis to a threshold to determine whether the result violates (e.g., exceeds, is less than, is outside of a range, etc.) the threshold or that the results of the analysis otherwise indicate that an action should be taken (e.g., based on historical data). In some embodiments, one or more of the thresholds may be statically defined by a user (e.g., via the data monitoring application 130), the settings of which may be stored in the settings database 406. However, the data analyzer 412 may be configured to determine or otherwise adjust one or more other thresholds in a dynamic fashion, as may be determined based on previously performed analyses and the results related thereto.

Other data either measured or observed may include age of the hive, length of beehive deployment, number of moves of the hive, other movement of the beehive, beehive mechanical structure information. Further, information can be gathered and calculations and other functions performed regarding crop yield and yield changes over time, and data regarding hive density, placement and any relationship with crop yield, calculations can be made and recommendations or instructions communicated regarding replacement of particular beehives, adding new beehives, removal of beehives, repositioning of beehives, and the like.

In another example, the data analyzer 412 may be configured to perform an image analysis on digital images captured (e.g., color, black and white, infrared, etc.) by one of the sensors 104. In an illustrative example, the data analysis engine 126 may be configured to analyze an image to determine an actual or estimated number of objects (e.g., eggs, types of bees, pollen, etc.) inside a particular beehive 102 based on one or more digital images received from a beehive sensor 106 embodied as an image sensor. It should be appreciated that the data analyzer 412 is configured to collect multiple data points for performing the analysis and/or updating a confidence level associated with a result of a particular analysis. In an illustrative example, the data analyzer 412 may be configured to analyze the movement of a queen bee such that liveness can be monitored. In such embodiments in which the data analyzer 412 determines the queen has not move for a particular duration of time such that it may be inferred that the queen bee's health has been compromised, the data analyzer 412 may be configured to analyze received digital images to validate a lack of movement, such as may be attributable to illness or death, rather than the transponder having fallen off the queen.

The action manager 414, which may be embodied as any type of firmware, hardware, software, circuitry, or combination thereof, is configured to determine which action is to be commenced or otherwise performed by the apiary data analysis engine 126, such as may be initiated upon a result of the analysis performed by the data analyzer 412 resulting in a determination that one or more thresholds have been violated. Such actions may include transmitting a message to a corresponding data monitoring application 130 and/or a user (e.g., via an email, text message, etc.), transmitting a command to one or more actuators 110 of the respective apiary 116, adjusting a time associated with a resource of the respective apiary 116 (e.g., an automated feeder schedule), adding a corresponding entry into a calendar (e.g., Google Calendar™, Outlook®, Thunderbird®, etc.) linked to the corresponding data monitoring application 130, etc. It should be appreciated that the action manager 414 may be configured to make the determination of which action to perform based on one or more settings corresponding to a user account associated with the apiary 116 being monitored. In some embodiments, the action settings and/or user account settings may be stored in the settings database 406.

The application interface manager 416, which may be embodied as any type of firmware, hardware, software, circuitry, or combination thereof, is configured to interface with the corresponding data monitoring applications 130. To do so, the application interface manager 416 is configured to establish a communication channel with the data analysis engine interface manager 308, which is usable to transmit and receive messages (e.g., network traffic, packets, messages, etc.) therebetween.

Additionally, the application interface manager 416 is configured to authenticate a user of the data monitoring application 130. To do so, the application interface manager 416 is configured to receive credentials from the user of the data monitoring application 130 and verify whether the user is an authorized user, such as may be performed based on a comparison between the received credentials and valid credentials of the user, which may be stored in the settings database 406. Further, the application interface manager 416 is configured to manage the access to data and/or the controls which are associated with the authenticated account.

In an illustrative example of the apiary data analysis engine 126 in use, a sensor 106 of a beehive 102 may be a weight sensor that is configured to detect a weight of at least a portion of the beehive 102. In such an embodiment, the weight sensor may transmit a weight value that is received by the sensor monitor 408, aggregated with historically received weight values, and analyzed by the data analyzer 412. The result of the analysis by the data analyzer 412 may result in a determination that the received weight value is greater than a weight threshold value, or is otherwise consistent with a weight threshold value that is indicative of a favorable time to extract honey from that beehive 102. Alternatively or additionally, weight loss for a sufficient period of time may indicate a deteriorating beehive health, which may result in a determination that the beehive needs to be checked by a beekeeper, or swapped with another beehive, moved, or some other action taken to improve the result.

Accordingly, as a function of the determination that the received weight value is greater than a weight threshold value and depending on the settings associated with an account corresponding to that beehive 102, the action manager 414 may be configured to perform one or more actions. Additionally, also dependent on the settings associated with an account corresponding to that beehive 102, the application interface manager may be configured to generate one or more messages for transmission to the respective data monitoring application that is usable to display one or more of a visual representation of the monitored data, the aggregated data, a result of the analysis of the monitored data and the aggregated data, and the action(s) to be taken, if applicable.

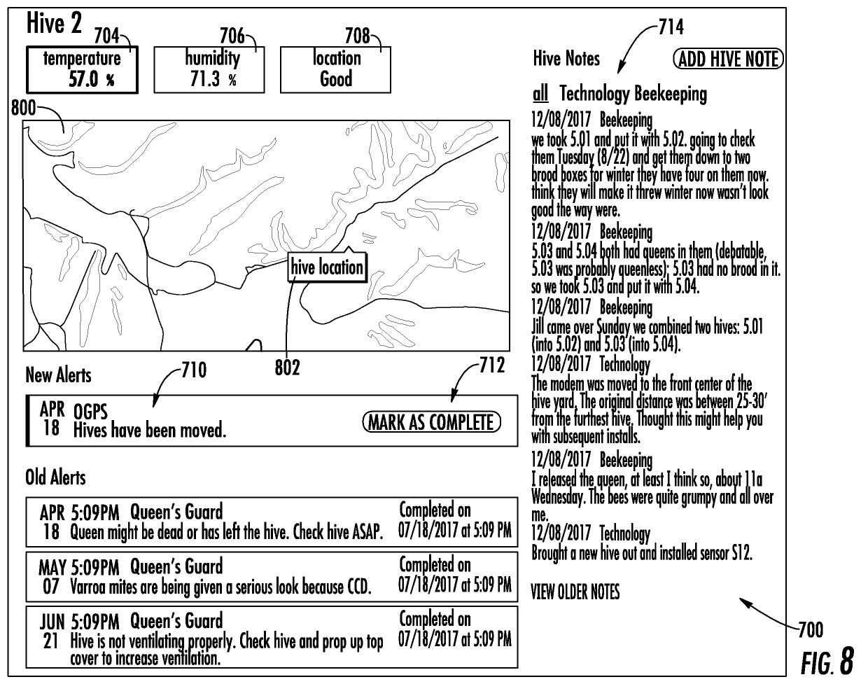

Referring now to FIG. 5, an illustrative method 500 is provided for monitoring and analyzing data of beehives (e.g., the beehives 102 of FIG. 1) in an apiary (e.g., the apiary 116 of FIG. 1) that may be performed by a remote data analysis computing device (e.g., the remote data analysis computing device 124 of FIG. 1), or more particularly by an apiary data analysis engine (e.g., the apiary data analysis engine 126 of FIG. 1) of the remote data analysis computing device. The method 500 begins in block 502, in which the apiary data analysis engine 126 determines whether sensor data has been received from one or more of the sensors 104 of the apiary 116 being monitored. As described previously, such data may correspond to a condition of a particular beehive 102, as may be received from one of the beehive sensors 106, or a condition of the apiary 116, as may be received from one of the environment sensors 108.

In block 504, the apiary data analysis engine 126 aggregates the received sensor data with previously received sensor data. In block 506, the apiary data analysis engine 126 analyzes the received sensor data and the aggregated sensor data. To do so, in some embodiments, in block 508, the apiary data analysis engine 126 is configured to analyze the data using one or more machine learning algorithms. Additionally or alternatively, in some embodiments, in block 510, the apiary data analysis engine 126 is configured to analyze the data using one or more regression analysis methods.

In block 512, the apiary data analysis engine 126 compares the results of the analysis to one or more thresholds. As described previously, the thresholds may be statically defined by a user (e.g., via the data monitoring application 130) or dynamically adjusted based on historical data (e.g., as may be determined based historical analysis performed and results related thereto). In block 514, the apiary data analysis engine 126 determines whether any thresholds have been violated or whether the results are otherwise indicative that an action should be taken. If the apiary data analysis engine 126 determines that an action should be taken in block 514, the method 500 advances to block 516.

In block 516, the apiary data analysis engine 126 performs one or more actions as a function of the comparison based on one or more settings of the apiary data analysis engine 126, as may be set by a user of a corresponding account via the data monitoring application 130. In some embodiments, in block 518, the apiary data analysis engine 126 may generate and transmit a message to the corresponding data monitoring application 130 (e.g., one or more instances of the data monitoring application 130 corresponding to the applicable account associated with the sensor data received in block 502. For example, in some embodiments, in block 520, the apiary data analysis engine 126 may be configured to display a notification in an interface of the data monitoring application 130. In another example, in some embodiments, in block 522, the apiary data analysis engine 126 may be configured to add an event to a calendar of the data monitoring application 130. It should be appreciated that, in some embodiments, the apiary data analysis engine 126 may be configured to add the calendar event to an external calendar application (e.g., Google Calendar™, Outlook®, Thunderbird®, etc.).

In some embodiments, in block 524, the apiary data analysis engine 126 may additionally or alternatively generate and transmit a command to a corresponding actuator (e.g., one of the actuators 110 of FIG. 1) that is usable by the receiving actuator to perform a particular operation (e.g., adjust a position of a mechanism attached to the actuator). Additionally or alternatively, in some embodiments, in block 526, the apiary data analysis engine 126 may generate and transmit an email to a corresponding email address of the user's account that includes information related to the action to be performed. It should be appreciated that, in some embodiments, the apiary data analysis engine 126 may be configured to provide a notification (e.g., via email, text, etc.) that indicates a state of the apiary 116, a beehive 102, and/or a particular one of the bees (e.g., the queen). In other embodiments, in block 528, the apiary data analysis engine 126 may additionally or alternatively generate and transmit a text message that includes information related to the action to be performed to a corresponding phone number of the user's account.

It should be appreciated that, in some embodiments, sensor data received from one or more other accounts, apiaries 116, beehives 102, etc., may be used to trigger an action. For example, a present state of a monitored beehive 102 (i.e., as determined based on the most recently received sensor data associated with that beehive 102) may indicate that an optimal time to extract honey from the monitored beehive 102 may be in two days; however, data of a similar beehive 102 in another apiary 116 associated with another user's account may indicate that now is an optimal time to extract the honey from the monitored beehive 102, such as may be based on weather patterns that affected the similar beehive 102 previously or are presently affecting the similar beehive 102 and are anticipated (e.g., based on the analysis performed in block 506) to have a similar effect on the monitored beehive 102.

It should be further appreciated that some information to be displayed (e.g., monitored sensor data, results of data analysis, etc.) and/or certain actions to be performed which are described herein may be queued for a particular user or account to which the actions correspond until a future point in time. For example, if that particular user or account associated therewith is not presently logged into the data monitoring application 130, such information may be queued until that user logs into the data monitoring application 130.

Data and Information Collection

| Data/Type of | | | | | | | | |
|---|---|---|---|---|---|---|---|---|
| Measurement | Outcome for Hive | | | | | Outcome for Beekeeper | | |
| From the beekeeper: | | | | | | | | |
| Standard | | 1. | Bees are more calm/less | | | | 3. | Less smoke needed |
| veils/masks to | | | aggressive/less stressed | | | | 4. | Increased beekeeper productivity |
| unify friendly | | 2. | | | | | | (beekeeper doesn't have to leave the |
| faces | | | | | | | | area) |
| | | | | | | | 5. | Can plan which hives to avoid/do last |
| | | 6. | Less volatile hive | | | | 7. | Beekeeper doesn't have to worry |
| | | | | | | | | about the hive |
| Beekeeper | Less bees caught = less dying bees | | | | | | | |
| clothing | | 1. | Bees less stressed | | | | 1. | Less smoke needed |
| | | | | | | | 2. | Increased beekeeper productivity |
| | | | | | | | | (beekeeper doesn't have to leave the |
| | | | | | | | | area) |
| | | | | | | | 3. | Beekeeper doesn't have to worry |
| | | | | | | | | about the hive |
| | | | | | | | 4. | Can plan which hives to do last/avoid |
| Food eaten | | 1. | Bad reaction (stinging or | | | | 3. | Less smoke needed |
| | | | aggression) | | | | 4. | Increased beekeeper productivity |
| | | 2. | Increased calmness | | | | | (beekeeper doesn't have to leave the |
| | | | | | | | | area) |
| | | | | | | Beekeeper doesn't have to worry about the | | |
| | | | | | | hive | | |
| Chemicals | | 1. | Harm bees with toxicity | | | | 1. | Lose a hive |
| interacted with | | | | | | | 2. | Loose hives around it |
| | | | | | | | 3. | Infected equipment |
| | | | | | | | 4. | Tainted honey and liability |
| | | | | | | | 5. | Tainted comb |
| | | | | | | | 6. | Increased cost |
| | | | | | | | 7. | Loss of productivity (honey and |
| | | | | | | | | laying) |
| | | 2. | Interferes with | | | Difficulty to gauge hive health and mood | | |
| | | | pheromones | | | | | |
| | | | | a. | Mood change | | | |
| | | | | b. | Ability to | | | |
| | | | | | communicate | | | |
| | | 3. | | | | | | |
| | | 4. | Ability to sense threat | | | Loss of a colony | | |
| Human | | 1. | Alters mood of hive | | | | 1. | Hard to gauge health |
| pheromones | | 2. | | | | | 2. | Hard to gauge normal moods |

| | | | | | | 3. | More smoke needed | |
|---|---|---|---|---|---|---|---|---|
| | | | | | | 4. | Effects beekeeper productivity | |
| | | 3. | Sends message to hive | | | 1. | Could optimize pheromones for | |
| | | | | | | | certain behavior in the hives | |
| | | | | | | 2. | Hive becomes conditioned to | |
| | | | | | | | beekeeper, making it easier over time | |
| Human noises | | 1. | Affects aggression (warning | | | 3. | Less smoke needed | |
| (sneezes, cough, | | | sign to bees) | | | 4. | Increased beekeeper productivity | |
| speech) | | 2. | | | | | (beekeeper doesn't have to leave the | |
| | | | | | | | area) | |
| | | | | | | 5. | Helps plan which hives to avoid/do | |
| | | | | | | | last | |
| | | 6. | Trained to expect them | | | 1. | Easier to interact | |
| | | | | | | 2. | More predictable | |
| | | | | | | 3. | | |
| Mood of | | 1. | | | | 1. | Less honey, less brood rearing | |
| Beekeeper | | 2. | Changes productivity | | | 2. | More attention needed | |
| | | 3. | Bees match the mood | | | 4. | Less smoke needed | |
| | | | | | | 5. | Increased beekeeper productivity | |
| | | | | | | | (beekeeper doesn't have to leave the | |
| | | | | | | | area) | |
| | | | | | | 6. | Helps plan which hives to avoid/do | |
| | | | | | | | last | |
| | | | | | | 7. | Chance the beekeeper might | |
| | | | | | | | overlook something | |
| Beekeeper Illness | | 8. | Bees match the mood | | | 9. | Less smoke needed | |
| | | | | | | 10. | Increased beekeeper productivity | |
| | | | | | | | (beekeeper doesn't have to leave the | |
| | | | | | | | area) | |
| | | | | | | 11. | Helps plan which hives to avoid/do | |
| | | | | | | | last | |
| | | | | | | 12. | Might overlook things in the hive | |
| | Illness transmitted to hive, might | | | | | 13. | Lose hive | |
| | alter pheromones, hive might | | | | | | | |
| | struggle, might lose productivity | | | | | | | |
| | and numbers | | | | | | | |
| Environmental: | | | | | | | | |
| Aerial infrared | 1. | Plants in the area | | | | 1. | Increased odds of honey | |
| and ultraviolet | 2. | | | | | 2. | Site selection | |
| | | | | | | 3. | Know about food and feeding year | |
| | | | | | | | round | |
| | 3. | Water sources | | | Site selection | | | |
| | 4. | Indicates food source as strong | | | | 1. | Site selection | |
| | | or weak | | | | 2. | When to feed | |
| | | | | | | 3. | How many hives can be placed in the | |
| | | | | | | | area | |
| Medicinal plants | Stronger defense and ability to | | | | | 1. | Lower hive loss | |
| available in area | combat against threats (including | | | | | 2. | Increase chance of survival | |
| | parasites and disease) | | | | | 3. | More success with other issues in the | |
| | | | | | | | hive | |
| | | | | | | 4. | Less hive checks needed | |
| | | | | | | 5. | Saves money and time | |
| | | | | | | 6. | Effects other hives | |
| Variety of food | 1. | Health and strength of hive | | | 1. | Taste of honey | | |
| sources in the | 2. | Vitamins that bees need | | | 2. | Color of honey | | |
| area (pollen and | | | | | 3. | Profitability of honey | | |
| nectar sources) | | | | | 4. | Times when food availability is scarce and | | |
| | | | | | | when type of feeding is needed (carbs or | | |
| | | | | | | protein) (action may be automated food | | |
| | | | | | | delivery or instruction, recommendation | | |
| | | | | | | for manual feeding) | | |
| | | | | | 5. | Peak times for extraction | | |
| Other pollinators | More competition | | | | | | 2. | Indicates good places to place |
| in the area | | | 1. | Less hive and | | | | bees |
| | | | | pollination productivity | | | 3. | Less chance of pollination |
| | | | | | | | | contracts |
| | | | | | | | 4. | More resources used to keep hive |
| | | | | | | | | alive |
| | | | | | | | 5. | Might mean less pesticides |
| | Increased disease | | | | | | 6. | Less honey revenue |
| | transmission = loss of production | | | | | | 7. | Might affect all hives |
| | Increased robbing | | | | | | 8. | Less honey |
| | | | | | | | 9. | Time spent (installing excluder, |
| | | | | | | | | checking hive, feeding |
| Trees in the area | 1. | More shade | | | | | | |
| | 2. | Cuts down on wind (Helps | | | | | 1. | More accurate readings |
| | | regulate temperature) | | | | | 2. | Increased success of hive and |
| | 3. | | | | | | | productivity |
| | 4. | | | | | | | |
| | 5. | Availability of food for honey, | | | | | 1. | Less $ spent on sugar |
| | | potentially year round | | | | | 2. | Less time feeding |
| | 6. | | | | | | 3. | More honey (more visits to |
| | | | | | | | | collect this) |
| | | | | | | | 4. | More valuable honey |
| | | | | | | | 5. | More money |

| Forest density | | 1. | More potential food | | 2. | Less $ spent on sugar | |
|---|---|---|---|---|---|---|---|
| | | | sources | | 3. | Less time feeding | |
| | | | | | 4. | More honey (more visits to collect | |
| | | | | | | this) | |
| | | | | | 5. | More valuable honey | |
| | | | | More money | | | |
| | | 6. | Easier to access food | | 7. | Collect more honey | |
| | | | sources | | 8. | More money | |
| Beekeeper hives | How fast food sources deplete | | | | | 1. | Less honey |
| nearby | | | | | | 2. | Interact with more beekeepers |

| | |
|---|---|
| a. | Political issues |
| b. | Increased robbing |

| | | | | 3. | Larger supply of honey = more |
|---|---|---|---|---|---|
| | | | | | buyer power |
| | Disease transmission | | | 4. | More treatments |
| | | | | 5. | More visits |
| | | | | 6. | Dependence on medication |
| | | | | 7. | Cross infection to other hives |
| | | | | 8. | Decreases survival |
| | More robbing | | | 9. | Less robbing |
| | | | | 10. | Time installing reducer |
| | | | | 11. | More time feeding |
| More of your | Less food | | | 12. | More time maintaining area |
| hives in one place | | | | 13. | Putting all of your eggs in one |
| | | | | | basket |
| | | | | 14. | Less time needed going from site |
| | | | | | to site. |
| Presence of other | Increase defense numbers of guard | | | 1. | Wasps might cause the |
| animals | bees, meaning less bees for | | | | beekeeper to be stressed |
| (predators | foraging and brood rearing | | | 2. | Hive at risk |
| seeking honey | | | | 3. | Make feeding difficult |
| and feeding on | | | | | |
| bees) | | | | | |
| Human density | | 1. | More pesticides | 1. | More time travelling to sites |
| (rural) | | 2. | Less diversity of food | 2. | Harder to get to |
| | | | sources (at least in Indiana) | 3. | Need diagnosis (time) |
| | | 3. | More diversity? | 4. | Immune system = hive at |
| | | | | | risk = treat more |
| | | | | 5. | Tainted honey |
| Human density | | 1. | Chemicals flows lawns | 1. | Easier to get around |
| (suburban) | | 2. | More diversity? Less | 2. | Traffic |
| | | | diversity? | 3. | Deal with upset neighbors |
| | | 3. | Air pollution, noise, might | 4. | Robbing |
| | | | affect hive health | | |
| Human density | | 1. | More pollution | 1. | Less resources used to replace |
| (urban) | | 2. | Less colonies in area, | | hive, hive better off |
| | | | decrease chance of disease | 2. | Less honey, low quality honey |
| | | | transmission | 3. | Need to feed more |
| | | 3. | More competing | 4. | Affects honey |

| | |
|---|---|
| | pollinators |
| 4. | Non natural food sources, |
| | might affect health |

| | | | | |
|---|---|---|---|---|
| Elevation | 1. | Ability for the queen to mate | 1. | Hive dies, need to get a queen |
| | 2. | Increased presence of wind | 2. | Easier for new queen to mate |
| | 3. | Decreased oxygen supply | 3. | More tiring, more rest needed |
| | 4. | Might affect performance, they | 4. | More gas needed |
| | | might sense storm differently | | |

| | | | | |
|---|---|---|---|---|
| Air pressure | 1. | Predict and observe weather | 3. | Predict when storms are coming |
| | | patterns and hive productivity | 4. | More $ and time |
| | 2. | Sensing threats | 5. | (sensing threats repeat here) |
| Latitude/longitude | 1. | Climate | 1. | Specific recommendations |
| | 2. | Bees have to adapt to climate | 2. | Regions of beekeeping |
| | | patterns | 3. | Might impede on the need to move bees |
| | 3. | Native and nonnative food | | around |
| | | sources | 4. | Lower pollination fees |
| | 4. | Magnetic field, need to point | 5. | Hurts reputation |
| | | them in a direction | 6. | (see food source) |

| | |
|---|---|
| 7. | Limits apiary design |
| 8. | Might not be feasible |
| 9. | Slope might be in the wrong direction, |

might limit where bees are placed

| | | | |
|---|---|---|---|
| Drone (flying) | | | What farms are nearby |
| Food availability | 1. | More health, more honey | Drives feeding schedule |

| | | | |
|---|---|---|---|
| 2. | Might increase competition | 3. | Less time |
| 3. | Difficulty for bees to | 4. | More money |
| | prioritize | 5. | Detract from honey |
| 4. | Might miss a bloom | 6. | Shorter season, less hive checks, |
| 5. | Do they go farther for | | more weather reliant |
| | better food (food sources | 7. | Don't get honey they want |
| | could be rated on variety of | 8. | Affect pollination contract |
| | factors or historical data | 9. | Might need to move bees for ore |
| | observed or accessed) | | nutrition |
| 6. | More foragers dying | 10. | Smaller honey crop, might not |
| 7. | Less efficient, less honey | | harvest honey |
| | produced in one day | 11. | Move hives for next year closer to |
| 8. | Less pollen brought in | | source |
| | | 12. | Might indicate better genes |

| | | | |
|---|---|---|---|
| Animal migration | 1. | Monarchs | Might indicate a good location with diversity |

| | |
|---|---|
| 2. | Increased competition for |
| | food sources |
| 3. | Birds |
| 4. | Different competition each |
| | year |

Chemical Interference

| | | | | |
|---|---|---|---|---|
| Pesticide use in | 1. | Death from toxicity | 1. | Might make beekeeper threat |
| surrounding area | 2. | Effect on the immune | 2. | Site selection to try to avoid them, |
| | | system (physio and | | would want land owners not spraying |
| | | psychological) | 3. | Adjust supply, add more hives |

| 3. | Might make food sources |
|---|---|
| | more or less attractive |
| 4. | Decreased competition |

| | |
|---|---|
| 5. | Application might affect |
| | bees differently |
| 6. | Used on one crop might |
| | affect other food sources in |
| | area on different plants |

| | | | | |
|---|---|---|---|---|
| Mosquito spray | 1. | death | 1. | needs to be notified when sprays |
| periods | | | | happen |
| | | | 2. | might need to make connection with |
| | | | | the local people in the area |
| Air quality | 1. | lifespan of bees | 1. | quality of honey |
| | 2. | difficulty to fly | 2. | more rest periods needed |
| | 3. | difficulty to breath | 3. | more dangerous uv rays |

| | |
|---|---|
| 4. | more grooming needed |
| 5. | more damgerous UV rays |

| | | | | |
|---|---|---|---|---|
| Water pollution | 1. | do they notice and avoid or | 1. | treatment and lossed hive |
| in area | | continue to consume | 2. | beekeeper needs to provide water |
| | 2. | toxicity affects | | source |
| | 3. | miught not be a water | 3. | a consideration in hive placement |

| | |
|---|---|
| | alternative |
| 4. | difference between salt |
| | and fresh water |
| 5. | treatment for human |
| | consumption |
| 6. | chlorinated water |
| 7. | puddles and chemicans on |
| | road |
| 8. | storm water runoff |
| 9. | flowing vs stagnant sources |

Climate/Weather factors

| Historical climate | Historically warm periods and the | | | 1. | Predict actions needed in hives | |
|---|---|---|---|---|---|---|
| data | hive's ability to prepare for winter | | | 2. | Drive hive placement | |
| | | | | 3. | Overwintering methods | |
| Fog | | 1. | Ability of bees to forage | | 1. | Difficult to drive |
| | | 2. | Difficulty defending hive if | | 2. | Might not be able to get to the |
| | | | not able to fly | | | bees |
| | | 3. | Hinder food source, as they | | 3. | Honey production |
| | | | might not be able to bring | | | |
| | | | it inside | | | |

| | | | | |
|---|---|---|---|---|
| Wind strength | 1. | How far they will forage | 1. | Honey production |
| (holders could be | 2. | Whether they forage at all | | |
| used to keep hive | 3. | Controlling temp within the | | |
| flush) | | hive | | |
| | 4. | Temp might be different in | | |
| | | different parts of the hive | | |
| | 5. | Is the priority to heat the hive | | |
| | | or get food, if so would one | | |

harm the other

| | |
|---|---|
| 6. | Loss of pollen and food sources |
| 7. | Pesticide drift |

| | | | |
|---|---|---|---|
| Wind direction | 1. | Direction the bees are | Hive placement consideration |

| | |
|---|---|
| | pushed towards |
| 2. | Might bring in different |
| | warm or cold air or |
| | different pressure |
| 3. | Type of wind |

| | | | | |
|---|---|---|---|---|
| Cloud cover | 1. | Ability to forage and bring | 1. | Money |
| | | in honey | 2. | Pollination contract issues |
| | 2. | Compare cloud cover in | 3. | Harder to visit hives |

| | | |
|---|---|---|
| similar climates | a. | Meaner, more time |

| 3. | Affects their ability to see | 4. | Cooler, more comfortable |
|---|---|---|---|
| | plants | 5. | Move hives |
| 4. | Not as much food available | 6. | Hive at risk |
| | from less sunlight | 7. | treat |
| 5. | Slower to get moving in the | | |
| | morning | | |
| 6. | How much does this affect | | |
| | things? | | |
| 7. | Increase fungus | | |

| | | | | |
|---|---|---|---|---|
| Temperature | 1. | affect hive foraging, | 1. | When to open hives |
| | | honey | 2. | Knowledge about the hive |
| | 2. | cant be productive | 3. | Changes when it is too hot |
| | 3. | have to cool hive, | 4. | If bearding and smoke if that hurts |
| | | detracts from other | | hive |
| | | productivity | 5. | Money |
| | 4. | propolis levels, wax | 6. | Lose hive |
| | | sagging, honey running | 7. | Affects when the beekeeper is |
| | 5. | moisture content | | available |
| | | within honey | 8. | Sweat affecting the hive |
| | 6. | increases mite and | 9. | Smell of a smelly beekeeper |
| | | other pest activity | 10. | Hot propolis —> easier |
| | | | 11. | Cold —> harder |
| | | | 12. | treat |

| | | | | |
|---|---|---|---|---|
| Shade/sunlight | 1. | Ability for control fungus | 1. | Where a beekeeper places a hive |
| levels | 2. | Changes productivity | 2. | Losing a hive |
| | 3. | More wax moths | 3. | When they do activities |
| | 4. | Don't wake up during the day | 4. | New foundation |

| | | |
|---|---|---|
| right away to get moving | a. | Lose productivity |

| | | | |
|---|---|---|---|
| 5. | Slower changes in productivity | 5. | Lose some honey crop |

| | | | |
|---|---|---|---|
| a. | Less time for honey | 6. | Maintenance time cleaning and |

| | | | |
|---|---|---|---|
| 6. | Ability to maintain | | treating, |
| | temperature levels | 7. | Time of day to visit bees |

| | | | |
|---|---|---|---|
| a. | Increases swarming | a. | Perspective of hives |

| | | |
|---|---|---|
| 7. | Overheat more in the sun | might be affected |

| | | | |
|---|---|---|---|
| a. | Cooling hive | 8. | Money |

| | | | |
|---|---|---|---|
| 8. | Shade: ability to stay warm | 9. | Harder to get in the hives |

| | | | |
|---|---|---|---|
| a. | Does the lack of leaves | 10. | Less time maintaining area |
| | make it easier for sun | | nearby |

| | | | |
|---|---|---|---|
| 9. | Propolis seals faster | 11. | Less time painting and |

| | | | |
|---|---|---|---|
| a. | Is it harder for bees to | | maintaining hives |
| | use propolis | 12. | Lose a hive |

| | |
|---|---|
| 10. | Trees nearby |

| | |
|---|---|
| a. | Drops branches, |
| | pesticides, dust, etc |
| b. | Animals nearby |

| | |
|---|---|
| 11. | More humid in shade |

| | | | | |
|---|---|---|---|---|
| Drought | 12. | Water availability | 13. | Beekeeper has to set our water |
| | 13. | Plants blooming, producing | 14. | Money |
| | | nectar and pollen | 15. | Feed more |

| | | | |
|---|---|---|---|
| a. | Honey yield | 16. | Losing hive |

| | |
|---|---|
| 14. | More dust more chances of |
| | pesticide drive |
| 15. | More time |

| | | | | |
|---|---|---|---|---|
| UV level | 1. | Increase visibility of flowers | 1. | Sunburnt, risks cancer |

| | | |
|---|---|---|
| and food sources | a. | Affects mood |

| | | | |
|---|---|---|---|
| a. | honey | 2. | Money |

| 2. | Sunburnt | 3. | Lose hive |
|---|---|---|---|
| 3. | Affects ability to see | 4. | Vision in hive |
| 4. | More pollen? | | |
| 5. | Weaker | | |

| | |
|---|---|
| a. | More burden on |
| | grooming and |
| | nurse bees |

| | | | | |
|---|---|---|---|---|
| Storms | 1. | Ability to collect honey | 1. | Honey production |
| | | (weight) | 2. | Drives when to beekeep |
| | 2. | Vibration changes in the hive | 3. | Money |
| | 3. | Pheromone changes inside the | 4. | Difficult to work with |
| | | hive | 5. | Change s beekeeper's perception of |
| | 4. | Pollen can get washed away | | how the hive is behaving |
| | | and entire flow gone | 6. | Beekeeper's ability to get out to the |
| | 5. | Changes behavior/prepare hive | | hives |
| | 6. | Affects strength | 7. | Can the beekeeper go beekeeping |
| | 7. | Are some storms more | | after a storm |
| | | stressful than others? | 8. | Easier to beekeep, less aggressive |

| | | | |
|---|---|---|---|
| a. | Longer storms | 9. | Beekeeper prepares for a storm |
| b. | Quick temperature | 10. | Effects when beekeeper smokes and |
| | drops | | then a storm also happens on the |

| | | |
|---|---|---|
| 8. | Natural disasters might be | hive |

devastating

| | |
|---|---|
| a. | Lightening effects |

| | |
|---|---|
| 9. | Do they change jobs when they |
| | cant forage |

| | | | | |
|---|---|---|---|---|
| | 10. | Does it get overcrowded | | |
| | 11. | After a storm, do they pick | | |
| | | back up? (if the storm | | |
| | | happened in the middle of the | | |
| | | day | | |
| | 12. | Electrical currents in air effects | | |
| | | their communication | | |
| | 13. | Is there a connection with the | | |
| | | earth's magnetic field | | |
| | 14. | What happened with the bees | | |
| | | in Florida and hurricane | | |
| | 15. | Do they forage from a tree, do | | |
| | | they forage from the ground | | |
| Length of daylight | 1. | Ability to forage and bring back | 1. | $ |
| | | honey | 2. | When to feed, super, harvest and |
| | 2. | Using phenology, bloom | | other activities |
| | | periods | 3. | How long the beekeeper can be in |
| | 3. | Foraging earlier/later | | the hive |
| | 4. | Hive to heat up more, might | 4. | Hive loss |
| | | need to cool hive | 5. | Consider where to put hives |
| | 5. | Might decrease nocturnal | 6. | Treatments |
| | | threats | 7. | More difficult to beekeep |

| | |
|---|---|
| a. | Increase stress and |
| | defensiveness |

| | |
|---|---|
| 6. | Flux and change of |
| | temperature |
| 7. | Bee orientation |
| 8. | Internal time clock moving |
| | might affect this |
| 9. | Decreases fungus growing in |
| | the hive |
| 10. | Increase pests |
| 11. | Ability to get honey |

During a hive visit

| Need to know | 1. | Bees reaction to smoke | 1. | Will be able to determine effect of |
|---|---|---|---|---|
| exactly what | | | | opening the hive |
| happens in the | | | 2. | Know whether the hive was opened |
| hive (that is | | | | |
| measurable) | | | | |
| when the hive is | | | | |
| opened | | | | |

| RFID on smoker | | 1. | What is the bees' behavior | 1. | Helps Bee Corp track hive visits for | | |
|---|---|---|---|---|---|---|---|
| | | | when they sense smoke? | | the beekeeper | | |
| | | 2. | Heat → rfid? | 2. | How long the beekeeper was there | | |
| | | 3. | How the hives around one | 3. | Affect perceptions of angry hives | | |
| | | | being smoked are affected | 4. | Less honey harvested | | |
| Music | | 1. | Frequencies, rhythms or | 1. | More pleasant to do work | | |
| Recording of | | | pitches affect their | 2. | Helps with their mood | | |
| peaceful hive | | | behavior or | 3. | productivity | | |
| noises | | | communication | | | | |
| | | 2. | Which type of speakers | | | | |
| | | 3. | Beneficial or harmful | | | | |
| Length of | 1. | How this affects hive | | 1. | effects productivity | | |
| intervention in | productivity/stress after leaving | | | 2. | added stress | | |
| each hive and | hive | | | 3. | more strenuous on the body | | |
| apiary as a whole | 2. | longer = more stressful | | 4. | accuracy they can see from bees | | |
| Maximum | | 1. | more smoke | | | a. | too short also may overlook |
| recommended | | 2. | temp regulation | | | | important things |
| time for various | | 3. | how much their | 5. | less honey or overall hive productivity | | |
| activities | | | productivity changes | 6. | swarms | | |
| (feeding/routine | | 4. | do scouts look for new | 7. | lose #s | | |
| check = short, | | | place to go | 8. | beekeeper will try to go less often to | | |
| treating for | | 5. | what's the queen doing | | be quicker | | |
| Varroa = longer) | | 6. | shorter hive visits better | | | | |
| | | | for bees | | | | |
| | | 7. | how long it takes them to | | | | |
| | | | get up to speed after | | | | |

| | | | | |
|---|---|---|---|---|
| Length of time in | 1. | hives nearby may be | 9. | bees may get used to scent |
| the apiary | | affected | 10. | productivity |
| | 2. | more stressed or less | 11. | beek job might be easier or worse |
| | | stressed with you there | 12. | less smoke needed |

| | |
|---|---|
| Smoke detector | Know when beekeeper is in hive |

| | | | | |
|---|---|---|---|---|
| Lime treatments | 1. | Small hive beetle levels | 1. | Might not work for pollination site |
| around the hive | 2. | Aroma have effect on | 2. | More or less sensitive to beekeeping |
| | | communication in the hive | 3. | Tainted honey |
| | 3. | Lime getting carried inside | 4. | Deformities |
| | | the hive, does this have an | 5. | Lost bees |
| | | effect | 6. | Lost equipment |
| | 4. | Affect water aupply | 7. | Lime might effect frame or feeder |
| Chemical | 1. | Does this affect the hive | 1. | Breeding poorly adapted bees |
| treatments | | adverselt | 2. | Toxic for beekeeper |
| | 2. | Gets rid of pest | 3. | Protective gear |
| | 3. | Life of queen | 4. | Frequency and time needed |
| | 4. | Long term affects | 5. | Timing of the application, might |

| | | | |
|---|---|---|---|
| a. | Do the chemicals | | happen on an unexpected dlow |
| | linger | 6. | Cause bees to swarm |
| b. | Cause | 7. | Behavior changes so beekeeper |
| | deformations | | doesn't recognize the natural state |
| c. | Next generation | 8. | Confusion on which treatment to use |
| | can fight or cant | 9. | Lose hive or not lose |
| | fight disease | 10. | Replace the queen |
| d. | Comb in the hive | 11. | Education needed on what the |
| e. | Honey | | problem is and the right treatment |

| | | | |
|---|---|---|---|
| 5. | Is the treatment | a. | What level of infection is |
| | contained? Does the | | necessary for treatment |

| | | | |
|---|---|---|---|
| | chemicals spread to other | 12. | weak hives, treat more, feed more, |
| | hives | | more attention needed |
| 6. | Chemicals interacting | 13. | purchase more comb and reinstall it |
| | (other treatments and | | and foundation |
| | pesticides | 14. | could lose honey crop |
| 7. | Impact on behavior | 15. | could get sued |
| 8. | How they affect the bees | 16. | knowing how much is too much, |
| | productivity | | affect the beekeeper's perception of |
| 9. | Zombie bees | | the hive |

| | | | | 17. | less pleasant to work with |
|---|---|---|---|---|---|
| Smoker fuel types | 1. | Harm caused to bees | 9. | How long the fuel lasts | |
| | 2. | How this affects hive | 10. | How expensive and easy it is to get | |
| | | productivity/stress after | 11. | Health problem | |
| | | leaving hive | 12. | Difficult or easy to light | |
| | 3. | Sends signal to hive | 13. | Condition the bees with different scents | |
| | 4. | Toxins released effect bees | | | |
| | 5. | Is it the smoke or the typical | | | |
| | | things used, is there an | | | |
| | | alternative | | | |
| | 6. | Eat their honey | | | |
| | 7. | Does ink/white | | | |
| | | paper/newspaper affect bees | | | |
| | 8. | Calm bees with scent or aoma | | | |

| | | | | |
|---|---|---|---|---|
| Size of smoker | 1. | Don't have hot smoke | 1. | How long the fuel lasts |
| used | | with bigger one | 2. | Cost difference |
| | 2. | Does it matter how big | 3. | Harder to clean |

| | |
|---|---|
| | the holes are |
| 3. | Bigger more powerful |
| | with smoke |

| | | | | |
|---|---|---|---|---|
| Beekeeper is | 1. | What happened inside the hive | 1. | Uncomfortable |
| stung (number of | | immediately | 2. | Stop beekeeping |
| stings, time | 2. | What happened after next visit | 3. | Makes you mellow |
| between stings) | 3. | Change in productivity | 4. | Stop working in the hive |
| | 4. | What is the reaction | 5. | Affects your day |
| | 5. | Which bees react | 6. | Masks the hive |
| | 6. | What do the drones do? | 7. | Probability of getting stung again |
| | 7. | Changes in hive nearby | 8. | Medical treatments may be necessary |
| | 8. | Is the pheromone universal | 9. | Is there a way to reverse it |

| | | | |
|---|---|---|---|
| a. | Is their scent stronger | 10. | Does it stay with the person who was |

| 9. | Bee smushed: does the bees | stung |
|---|---|---|
| | give the pheromone or is it | |
| | natural when the stinger is | |
| | removed? Is there a difference | |
| | in the two | |
| 10. | Sting happens elsewhere, are | |

| | | the bees on edge | | | | |
|---|---|---|---|---|---|---|
| | 11. | Is the pheromone different | | | | |
| | | with different animals | | | | |
| | 12. | What happens if the bee | | | | |
| | | loosed his stinger anywat | | | | |
| Footage of hive | | | 1. | How many bees are in | 1. | Great for record keeping |
| visit | | | | a frame | 2. | When to put on a new super |
| | | | 2. | Software that could | 3. | Compare footage over time |
| | | | | gague the number of | 4. | Sense of speed in the hive |
| | | | | bees | 5. | Able to go back and inspect hive in |
| | | | 3. | % brood filled up | | detail |
| | | | | | 6. | Estimate mite levels |
| | | | | | 7. | Different honey flows happening |
| | | | | | 8. | How long honey has been capped |
| | | | | | 9. | See the life cycle of the bee |
| | | | | | 10. | Finding queen |
| | | | | | 11. | Someone live viewing to look for |
| | | | | | | specific problems |
| | | | | | 12. | Beekeeper support |
| | | | | | 13. | Online classes |
| | | | | | 14. | Clips for classes |
| Alternative | | | 1. | Interferes less with the | 1. | Might be wrongly targeted |
| disease | | | | chemicals with | 2. | Less risk on contaminated honey |
| treatments | | | | pheromones | | |
| | | | 2. | Less toxic | | |
| | | | 3. | Not effective | | |

| | | | | |
|---|---|---|---|---|
| Pheromones | 1. | Stress levels after | 1. | Depends on whether they can be |
| | | intervention | | detected by humans |
| | 2. | Pheromone is specific to | 2. | Used to manipulate the hive |

| | | |
|---|---|---|
| disease or disruption | a. | Threatening presence, etc. |

| | |
|---|---|
| a. | Either it is different |
| b. | Whether there is |
| | anything that |
| | blocks them |
| | (parasite or |
| | disease) |

| | |
|---|---|
| 3. | What causes the |
| | pheromones to be emitted |
| 4. | Communicate with other |
| | bees, is it just alarms |
| 5. | Can they overpower the |
| | queen |
| 6. | Difference between active |
| | and passive pheromones |
| 7. | Where food sources are |
| 8. | Conditions of the hive |

Food:

| Time of day bees | 1. | Whether they forage less | | | 6. | Timed delayed sensor to help feed |
|---|---|---|---|---|---|---|
| are fed | | | a. | Can this help them | | bees |

| 2. | Do they work at night? If so | 7. | Less honey and money | | |
|---|---|---|---|---|---|
| | then feeding can be | 8. | Less valuable honey | | |
| | strategic | 9. | Less nutritious honey | | |
| 3. | When do they eat | 10. | Weaker genetics over time | | |
| 4. | Number of foragers that | 11. | More food overall for the bees | | |
| | leave | 12. | Time of day that is optimal to feed | | |
| 5. | Less nutritious honey | | | a. | May be more receptive to it |

in evening

| | | | | |
|---|---|---|---|---|
| Vitamin | 1. | Could breed them to be | 1. | Becomes a necessity |
| supplements fed | | reliant | 2. | Genetic trait good or bad |
| to bees | 2. | Less resistant to health | 3. | More money spent |
| | | problems, or more | 4. | Higher chance of hive success |

| | |
|---|---|
| 3. | Enhance nutritional value |
| | of honey |
| 4. | Stronger bees in short term |

| | | | | |
|---|---|---|---|---|
| Food type fed to | 1. | Certain types of sugar harm | 1. | Could take out frames and store them |
| hive (agave, | | the hive | | for later feeding (like after a spray) |
| sugar, maple | 2. | Consistence with water- | 2. | Does this get into honey |
| syrup, honey) | | what level | 3. | Differing costs |

| | | | |
|---|---|---|---|
| a. | Are certain feeds | 4. | Different time spent prepping |

better at different
times of the year

| | | | | |
|---|---|---|---|---|
| Overwinter | 1. | Survivability | 1. | Might save hive |
| feeding | 2. | How frequent they need it | 2. | Flexible in winter to visit hives |
| | 3. | Do they need it year round | 3. | Cost and time spent doing this |

| | or do they take when they | | | a. | Time spent checking it, |
|---|---|---|---|---|---|
| | need | | | | depending on type |
| 4. | Sense of conserving? | 4. | Add supers or switch out frames of | | |
| 5. | Prevent starvation | | honey | | |
| 6. | Stronger in spring and | | | | |
| | could swarm | | | | |
| 7. | Regularly, though winter, | | | | |
| | getting out when warm | | | | |
| 8. | In warm weather when | | | | |
| | they look for food | | | | |
| | (optionally plant for warm | | | | |
| | days) | | | | |
| 9. | Length that it is inside the | | | | |
| | hive | | | | |
| 10. | Which type is better | | | | |

Genetics:

| | | | | |
|---|---|---|---|---|
| Type of bee | 1. | Overwintering success | 1. | Aggressiveness (stinging) |
| (Italian, Russian, | 2. | Honey production | 2. | Price buying and selling hives |
| etc.) | 3. | Ability to defend hive from | 3. | How many times you treat |
| | | robbing and other threats | 4. | Who your customer is |
| | 4. | More mite and disease | 5. | Pollination effectiveness |
| | | resistant | 6. | Honey production |
| | 5. | Better at reproduction | 7. | Affect breeding practices |
| | 6. | Queen difference in | 8. | Time spent in the hive |
| | | acceptability into hive and | 9. | Whether you want to check on them |
| | | lifespan | | or not |
| Location queen | 1. | Success of eggs in | 1. | Cost |
| was bred | | environment | 2. | How easy it is to get it |
| | 2. | Better more effective | 3. | Could save a hive |
| | | mating or a queen who has | 4. | Chance of requeening success |
| | | been conditioned to the | 5. | |
| | | climate | | |
| | 3. | Overwintering success | | |
| | 4. | Elevation: does this chance | | |
| | | her distance in the air, or is | | |
| | | it based on the distance to | | |
| | | the ground | | |

| | |
|---|---|
| a. | Does this increase |
| | predators |

| Who the breeder | 1. | Mite resistance | | 1. | Price |
|---|---|---|---|---|---|
| is | 2. | Experience in years | | 2. | Reputation and relationships |
| | 3. | Traits the beek favors | | 3. | Resources used |
| | 4. | Overwintering success with | | 4. | Ability to give customer service |
| | | queens | | | |
| | 5. | Methods they use to | | | |
| | | identify traits (purdue and | | | |
| | | insemination, good record | | | |
| | | keeping, Beekeeper | | | |
| | | memory) | | | |
| Which traits were | | 1. | Aggressiveness | 1. | Get to pick what you prefer |
| selected | | 2. | Resistance | 2. | Price |
| | | 3. | Production | 3. | Aggressive queen + tame hive = will |
| | | 4. | How long they have | | this work? |
| | | | hone it for | | |

| Wing span & | | 1. | Foraging distance & ability | | 1. | Honey crop |
|---|---|---|---|---|---|---|
| thickness | | | to seek out plants | | 2. | More valuable honey |
| | | 2. | Mating distance and height | | 3. | survivability |
| | | 3. | Energy used in flying | | | |
| | | 4. | How much they can carry | | | |
| | | 5. | How long they live | | | |
| | | 6. | Food they eat/plants | | | |
| | | 7. | Capacity in hive: super | | | |
| | | | more often? | | | |
| | | 8. | Cool the hive | | | |
| Bee Color | | 1. | different from standard, | | | |
| | | | might affect their | | | |
| | | | aggression | | | |
| | | 2. | heating and cooling | | | |
| | | 3. | Italians might be more of a | | | |
| | | | warning to other animals | | | |
| | | 4. | Might affect how bees see | | | |
| | | | each other | | | |
| Splits | 1. | Survival rates | | | 1. | Timing, need to get this right |
| | 2. | Difference in late splits vs | | | 2. | Amount of equipment purchased |
| | | spring splits in survival and | | | 3. | Save hives and revenue |
| | | productivity | | | 4. | Which one do you move aeay |
| | 3. | Honey production (different | | | | |
| | | between old and new hive?) | | | | |
| | 4. | If need to build out | | | | |
| | | comb = reduced production | | | | |
| | 5. | Overwintering success for both | | | | |
| | | hives | | | | |
| Emergency queen | 1. | Survival rates | | | | |
| | 2. | Acceptance rates | | | | |
| Mail order queen | 1. | Survival rates | | | | |
| | 2. | Acceptance rates | | | | |
| Creating | | | | With certain equipment? Prevent Dirt | | |
| grooming | | | | entering hive? | | |
| behavior | | | | | | |
| Cleanliness of | | | | | | |
| hive | | | | | | |
| RFID on queen | How fast she goes | | | | | |
| About the hive: | | | | | | |
| Queen tracking | How fast she lays eggs | | | | | |
| with RFID | Mating behavior (elevation mating | | | | | |
| Consider Global | occurs, environmental factors, etc.) | | | | | |
| Initiative for | | | | | | |
| Honeybee Health | | | | | | |
| Propolis levels | Pest levels in the hive | | | | | |
| | Number of foragers | | | | | |
| Gases inside the | | | | | | |
| hive | | | | | | |
| Weight | 1. | Population size | | 1. | When to start feeding | |
| | 2. | Proportions of workers to | | 2. | When to take honey off | |
| | | drones | | 3. | When to split hives | |
| | 3. | Honey production | | | | |
| Cause of death | | | | How to best treat equipment upon death | | |
| Other hives | | | | | | |
| located in the | | | | | | |
| apiary | | | | | | |

| Varroa mite load | Alert beekeeper when it reached a critical |
|---|---|
| (sticky board, | point |
| other testing | |
| methods) | |

| Small hive beetle | | | | Alert beekeeper when it reaches a critical | | |
|---|---|---|---|---|---|---|
| levels | | | | point | | |
| Wax moth levels | | | | Alert beekeeper when it reaches a critical | | |
| | | | | point | | |
| Location | | | | Optimal travel routes | | |
| Pheromones | 1. | Intruders | | | | |
| | 2. | Supercedure | | | | |
| | 3. | New queen | | | | |
| | | acceptance/resistance | | | | |
| Hive splits | 1. | Did they survive | | | | |
| | 2. | Compare late splits vs spring | | | | |
| | | splits | | | | |
| Emergency | | | | Look at ordered queens and transferred | | |
| queens | | | | frames | | |
| Hive is moved | | 1. | Lossed foragers, | | 1. | Hive and money |
| | | | production | | 2. | Less valuable pollination contract |
| | | 2. | Need orientation | | 3. | Less pleasant to work with, more |
| | | 3. | Increased stress | | | aggressive |
| | | 4. | New climate, pests, | | 4. | Tainted honey and usability |
| | | | diseases | | 5. | Cant split hives |
| | | 5. | New food sources and | | 6. | Decreased honey production and |
| | | | pesticides (does previous | | | overall value |
| | | | immunity translate to a | | | |
| | | | new one) | | | |
| | | 6. | Less resistant to threats in | | | |
| | | | both areas | | | |
| | | 7. | Might affect brood raising | | | |

| Bee Proportions: | | |
|---|---|---|
| Number of | 1. | Mating success |
| drones | 2. | Honey consumption |

| | |
|---|---|
| Proportion of | Number of new bees/breeding rate |
| new bees (“hairy | |
| bees” | |
| Proportion of | Preparing for winter |
| winter bees (“fat | |
| bees”) | |

Comb:

| Age of the comb | 1. | Whether it is more conducive | Alert that the beekeeper should replace comb |
|---|---|---|---|
| | | to the spread of disease | |
| | 2. | Whether it retains pesticides | |
| Comb size/Bee | Varroa mite levels in hive | | |
| size (correlated) | | | |
| Size of | 1. | Mating success | |
| drones/Comb | 2. | Varroa mite levels | |
| size | | | |

| | | |
|---|---|---|
| Presence of brace | | |
| comb | | |
| Amount of drone | | |
| brood in the hive | | |
| Frame weight | 1. | Population |
| | 2. | Capped cells |
| | 3. | Honey |
| Laying pattern | 1. | Strength of the queen |
| | 2. | Diseases |
| | 3. | Queens movement throughout |
| | | the hive |
| | 4. | Age of the queen |

Equipment:

| Preventing dirt | | | |
|---|---|---|---|
| being brought in | | | |
| Wrapping during | Survivability | | |
| winter | | | |
| Bottom board | | | |
| style | | | |
| Ventilation | Ability to control humidity in winter | | |
| Carbon filters | | | |
| Hive foundation | Small hive beetle levels | | |
| used | | | |
| Age of equipment | Probability of holding disease | | Time to repair |
| Number of | | | |
| repairs to | | | |
| equipment | | | |
| Type of comb | How quickly it is filled out | | |
| foundation | | | |
| Frames used in | | | Honey harvest |
| honey super | | | |
| Size of super | How quickly is it filled out? | | |
| | How far up does the queen travel? | | |
| Number of supers | Survivability | | |
| used | | | |
| Slope of hive | Ability for water to drain | | |
| Entrance | | | |
| direction | | | |
| Position of | | | |
| frames (honey | | | |
| outer, brood | | | |
| inner) | | | |
| Infrared camera | | | |
| Style of hive | | | |
| Color of hive | 1. | Ability of bees to spot the hive | |
| | 2. | Amount it reflects heat | |

| | |
|---|---|
| Type of top cover | Amount of ventilation |
| used | |

Factors used in pollination contracts:

| Rate of foraging | 1. | Health/strength of hive | 1. | Determines prices |
|---|---|---|---|---|
| (RFID on | 2. | Increased chance of survival | 2. | Increases negotiation power for the |
| foragers) | 3. | How fast they are | | beekeeper |
| | 4. | How much time they spend | | |
| | outside the hive | | | |
| | 5. | Genetic trait? | | |
| Transportation | | | | |
| style | | | | |
| Transportation | | | | |
| distance | | | | |

| | |
|---|---|
| Acceleration or | Whether this negatively affects |
| velocity reached | them |
| Knowledge about | Health of hive |
| a hive when in | Whether they're effective in |
| holding yards | calming/acclimating bees, feeding |
| | performed, distance between |
| | holding yard and pollination site |

Other:

| | |
|---|---|
| Honey prices | Optimal times to sell honey |

While the invention has been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

What is claimed is:

1. A method for monitoring and analyzing data of beehives in an apiary, the method comprising:
receiving, by a data analysis computing device, sensor data from one of a plurality of sensors associated with a beehive of a plurality of beehives of the apiary;
analyzing, by the remote data analysis computing device, the received sensor data;
determining, by the remote data analysis computing device, whether to perform an action relative to the beehive as a function of a result of the analysis.

2. The method of claim 1 further comprising performing, by the remote data analysis computing device and in response to a determination the action is to be performed, the action relative to the beehive.

3. The method of claim 2. wherein data is received at the analysis computing device remote from the sensors.

4. The method of claim 1, wherein analyzing the sensor data comprises to analyze the data using one or more machine learning algorithms.

5. The method of claim 1, wherein analyzing the sensor data comprises to analyze the data using one or more regression analysis methods.

6. The method of claim 1, further comprising aggregating, by the remote data analysis computing device, the received data with previously received sensor data from at least a portion of the plurality of sensors, wherein analyzing the received data includes analyzing the aggregated data.

7. The method of claim 1, further comprising comparing, by the remote data analysis computing device, the result of the analysis to one or more threshold values, wherein determining whether to perform the action as a function of the result of the analysis comprises determining whether to perform the action as a function of the comparison.

8. The method of claim 7, wherein performing the action in response to a determination the action is to be performed comprises performing the action in response to a determination that the result of the analysis violates one or more threshold values.

9. The method of claim 1, wherein performing the action comprises transmitting a message to a corresponding data monitoring application, wherein the message includes information related to the action that is usable to display to a user of the corresponding data monitoring application.

10. The method of claim 1, wherein performing the action comprises transmitting a command to an actuator of the apiary, wherein the command is usable by the actuator to adjust a position of a mechanism attached to the actuator.

11. The method of claim 1, wherein performing the action comprises transmitting an email to an email address, wherein the email includes information related to the action that is usable to indicate to notify a user of the action.

12. The method of claim 1, wherein performing the action comprises transmitting a text message to a phone number, wherein the text message includes information related to the action that is usable to notify a user of the action.

13. A communications and control system for monitoring and making decisions regarding a plurality of beehives comprising:
A plurality of beehives;
At least one beehive sensor associated with each beehive for measuring data at each beehive and/or at least one environment sensor associated with each beehive for measuring the environment around the beehive(s);
A data communications and collection device for collecting data from the at least one beehive sensor; and
A processor for performing one or more functions on the data collected and using a result of the one or more functions to communicate a signal regarding an action on the at least one beehive.

14. The system of claim 13 wherein the one or more functions includes one or more of the following:
determining average weight change over time of the one or more beehives
determining an activity level of the hive using one or more of the following:
Decibel level,
IR movement measures,
Visual movement measures,
Near continuous weight change measures.
and the signal is selected from the group comprising:
a signal to open a feed door,
a signal to open a smoke hatch,
a signal to send a message regarding hive movement or recommend new hive placement,
a signal to generate a report to a user at a user device,
a signal to take another measurement with one or more sensor(s),
a signal to generate a warning to a user at a user device,
a signal to generate a status check request to a user device,
a signal to activate the playing of an audio recording,
a signal to activate or move a wind screen,
a signal to activate or move a sun screen,
a signal to activate or deactivate a heater,
a signal to activate or deactivate a fan or air conditioning unit.

15. The system of claim 13, wherein the at least one beehive sensor measures or observes one or more of the following:
temperature(s) at different points on or in the hive,
gas (such as oxygen, carbon monoxide, and/or carbon dioxide gases) or other material or toxin levels,
infrared or other wavelength information,
weight and/or mass,
activity level of bees in, entering and/or leaving, the beehive,
decibel or other noise level measures,
humidity levels,
pressure,
activity such as may be measure by movement or another visual measure, GPS or similar data regarding physical location,
altitude,
orientation or relative orientation,
other information or data regarding the beehive, bees, their activity, health, stress
or other beehive effects.

16. The system of claim 13, wherein at least one environment sensor measures or observes one or more of the following:
temperature(s) at different points on or in the hive,
gas (such as oxygen, carbon monoxide, and/or carbon dioxide gases) or other material or toxin levels,
decibel or other noise level measures,
humidity levels,
pressure, GPS or similar data regarding physical location,
altitude,
wind speed and/or direction,
precipitation type and amount.

17. The system of claim 13, further including a communications input or collection device to gather crop information and data selected from the group comprising crop type and related data, plant location, plant density, crop yield information whether aggregate or by location in an orchard, field or plot), pesticide, fertilizer, and/or irrigation type, quantity and rate information, planting and harvest information, and other agricultural information.
